(19) United States
(12) Patent Application Publication
(10) Pub. No.: US 2011/0124650 A1
(43) Pub. Date: May 26, 2011

(54) STABLE CRYSTALLINE SALTS OF ANTIFOLATE COMPOUNDS

(75) Inventors: Harish K. Pimplaskar, Charlotte, NC (US); Mikhail Lebedev, Sodertalje (SE); Karol Horvath, Sodertalje (SE)

(73) Assignee: Chelsea Therapeutics, Inc.

(21) Appl. No.: 12/831,617

(22) Filed: Jul. 7, 2010

Related U.S. Application Data
(60) Provisional application No. 61/223,888, filed on Jul. 8, 2009.

(51) Int. Cl. A61K 31/519; C07D 239/95; A61K 31/517; A61P 19/02; A61P 29/00; A61P 11/06; A61P 35/00

(52) U.S. Cl. 514/249; 544/291; 514/266.4

(57) ABSTRACT

The present invention provides stable crystalline polymorphic forms of antifolate compounds, particularly (S)-2-{4-[2-(2,4-diamino-quinazolin-6-yl)-ethyl]-benzoylamino}-4-methylene-pentanedioic acid, dipotassium salt, and methods of preparation thereof The polymorphs may be in the form of hydrates. The invention further provides pharmaceutical compositions comprising the polymorphs and methods of treatment using the polymorphs. The polymorphs are useful in the treatment of multiple conditions, including abnormal cell proliferation, inflammatory diseases, asthma, and arthritis, and the polymorphs may be administered alone in or combination with on or more further active agents.

### CROSS-REFERENCE TO RELATED APPLICATION

The present patent application claims priority to U.S. Patent Application No. 61/223,888, filed Jul. 8, 2009, the disclosure of which is incorporated herein by reference in its entirety.

### FIELD OF THE INVENTION

The present application is directed to polymorphs of antifolate compounds, methods for the preparation thereof, as well as compositions comprising the polymorphs, and methods of treatment using the polymorphs.

### BACKGROUND

Folic acid is a water-soluble B vitamin known by the systematic name N-[4(2-amino-4-hydroxy-pteridin-6-ylmethylamino)-benzoyl]-L(+)-glutamic acid and having the following structure.

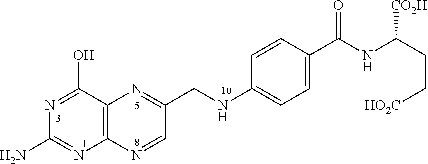

As seen in the above formula, the folic acid structure can generally be described as being formed of a pteridine ring, a para-aminobenzoic acid moiety, and a glutamate moiety. Folic acid and its derivatives are necessary for metabolism and growth, particularly participating in the body's synthesis of thymidylate, amino acids, and purines. Derivatives of folic acid, such as naturally occurring folates, are known to have biochemical effects comparable to folic acid. Folic acid is known to be derivatized via hydrogenation, such as at the 1,4-diazine ring, or being methylated, formaldehydylated, or bridged, wherein substitution is generally at the N⁵ or N¹⁰ positions. Folates have been studied for efficacy in various uses including reduction in severity or incidence of birth defects, heart disease, stroke, memory loss, and age-related dementia.

Antifolate compounds, like folates, are structurally similar to folic acid; however, antifolate compounds function to disrupt folic acid metabolism. A review of antifolates is provided by Takamoto (1996) The Oncologist, 1:68-81, which is incorporated herein by reference. One specific group of antifolates, the so-called “classical antifolates,” is characterized by the presence of a folic acid p-aminobenzoylglutamic acid side chain, or a derivative of that side chain. Another group of antifolates, the so-called “nonclassical antifolates,” are characterized by the specific absence of the p-aminobenzoylglutamic group. Because antifolates have a physiological effect that is opposite the effect of folic acid, antifolates have been shown to exhibit useful physiological functions, such as the ability to destroy cancer cells by causing apoptosis.

Folate monoglutamylates and antifolate monoglutamylates are transported through cell membranes either in reduced form or unreduced form by carriers specific to those respective forms. Expression of these transport systems varies with cell type and cell growth conditions. After entering cells most folates, and many antifolates, are modified by polyglutamylation, wherein one glutamate residue is linked to a second glutamate residue at the a carboxy group via a peptide bond. This leads to formation of poly-L-γ-glutamylates, usually by addition of three to six glutamate residues. Enzymes that act on folates have a higher affinity for the polyglutamylated forms. Therefore, polyglutamylated folates generally exhibit a longer retention time within the cell.

An intact folate enzyme pathway is important to maintain de novo synthesis of the building blocks of DNA, as well as many important amino acids. Antifolate targets include the various enzymes involved in folate metabolism, including (i) dihydrofolate reductase (DHFR); (ii) thymidylate synthase (TS); (iii) folylpolyglutamyl synthase; and (iv) glycinamide ribonucleotide transformylase (GARFT) and aminoimidazole carboxamide ribonucleotide transformylase (AICART).

The reduced folate carrier (RFC), which is a transmembrane glycoprotein, plays an active role in the folate pathway transporting reduced folate into mammalian cells via the carrier mediated mechanism (as opposed to the receptor mediated mechanism). The RFC also transports antifolates, such as methotrexate. Thus, mediating the ability of RFC to function can affect the ability of cells to uptake reduced folates.

Polyglutamylated folates can function as enzyme cofactors, whereas polyglutamylated antifolates generally function as enzyme inhibitors. Moreover, interference with folate metabolism prevents de novo synthesis of DNA and some amino acids, thereby enabling antifolate selective cytotoxicity. Methotrexate, the structure of which is provided below, is one antifolate that has shown use in cancer treatment, particularly treatment of acute leukemia, non-Hodgkin's lymphoma, breast cancer, head and neck cancer, choriocarcinoma, osteogenic sarcoma, and bladder cancer.

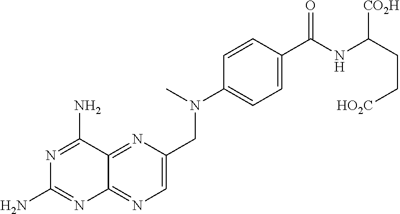

Nair et al. (J. Med. Chem. (1991) 34:222-227), incorporated herein by reference, demonstrated that polyglutamylation of classical antifolates was not essential for anti-tumor activity and may even be undesirable in that polyglutamylation can lead to a loss of drug pharmacological activity and target specificity. This was followed by the discovery of numerous nonpolyglutamylatable classical antifolates. See Nair et al. (1998) Proc. Amer. Assoc. Cancer Research 39:431, which is incorporated herein by reference. One particular group of nonpolyglutamylatable antifolates are characterized by a methylidene group (i.e., a ═CH₂ substituent) at the 4-position of the glutamate moiety. The presence of this chemical group has been shown to affect biological activity of the antifolate compound. See Nair et al. (1996) Cellular Pharmacology 3:29, which is incorporated herein by reference.

Further folic acid derivatives have also been studied in the search for antifolates with increased metabolic stability allowing for smaller doses and less frequent patient administration. For example, a dideaza (i.e., quinazoline-based) analog has been shown to avoid physiological hydroxylation on the pteridine ring system. Furthermore, replacement of the secondary amine nitrogen atom with an optionally substituted carbon atom has been shown to protect neighboring bonds from physiological cleavage.

One example of an antifolate having carbon replacement of the secondary amine nitrogen is 4-amino-4-deoxy-10-deazapteroyl-γ-methyleneglutamic acid—more commonly referred to as MDAM—the structure of which is provided below.

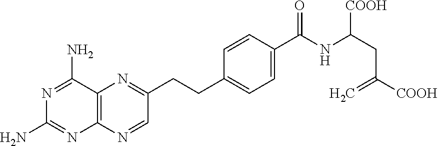

The L-enantiomer of MDAM has been shown to exhibit increased physiological activity.

See U.S. Pat. No. 5,550,128, which is incorporated herein by reference. Another example of a classical antifolate designed for metabolic stability is ZD1694, which is shown below.

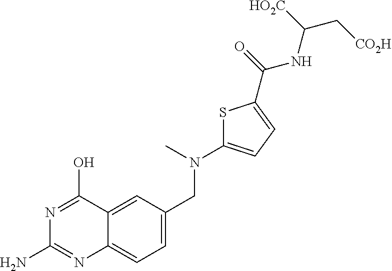

A group of antifolate compounds according to the structure shown below combines several of the molecular features described above, and this group of compounds is known by the names MobileTrexate, Mobile Trex, Mobiltrex, or M-Trex.

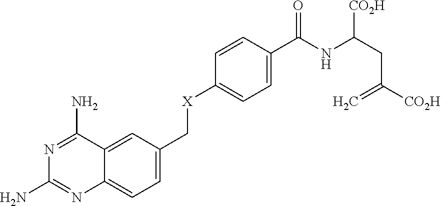

As shown in the formula above, this group of compounds encompasses M-Trex, wherein X can be CH₂, CHCH₃, CH(CH₂CH₃), NH, or NCH_{3.}

The effectiveness of antifolates as pharmaceutical compounds arises from other factors in addition to metabolic inertness, as described above. The multiple enzymes involved in folic acid metabolism within the body present a choice of inhibition targets for antifolates. In other words, it is possible for antifolates to vary as to which enzyme(s) they inhibit. For example, some antifolates inhibit primarily dihydrofolate reductase (DHFR), while other antifolates inhibit primarily thymidylate synthase (TS), glycinamide ribonucleotide formyltransferase (GARFT), or aminoimidazole carboxamide ribonucleotide transformylase, while still other antifolates inhibit combinations of these enzymes.

While antifolates are useful for treating a variety of conditions, it can be difficult to provide antifolate compounds in a stable form, particularly a form that can be incorporated into pharmaceutical compositions.

### SUMMARY OF THE INVENTION

It has now been determined according to the present invention that specific compounds having antifolate activity can be provided in stable polymorphic forms that have distinctly different physical properties. In particular, the invention provides stable polymorphic forms of the compound (S)-2-{4-[2-(2,4-diamino-quinazolin-6-yl)-ethyl]-benzoylamino}-4-methylene-pentanedioic acid, dipotassium salt—the compound of Formula (1). The invention further provides various stable crystalline pseudopolymorphic forms of the compound, such as hydrates.

In one embodiment, the invention provides a crystalline polymorph of the compound of Formula (1),

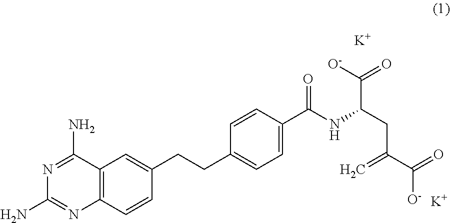

wherein the polymorph is designated as polymorph Form Ia. The polymorph Form Ia can be characterized by having one or more of the approximate X-ray powder diffraction interplanar spacing peaks selected from the group consisting of 4.946, 7.118, 7.785, 8.238, 9.229, 9.822, 13.4000, 15.271, 15.658, 16.128, 16.459, 17.286, 18.088, 17.452, 18.889, 19.490, 19.837, 21.456, 22.658, 23.168, 23.811, 24.691, 28.436, and 29.609. Form Ia also can be characterized by having a specific X-ray powder diffraction pattern graph as illustrated herein.

In another embodiment, the invention provides a crystalline polymorph of the compound of Formula (1), wherein the polymorph is designated as polymorph Form Ib. The polymorph Form Ib can be characterized by having one or more of the approximate X-ray powder diffraction interplanar spacing peaks selected from the group consisting of 4.811, 8.316, 9.542, 10.047, 13.189, 14.946, 15.973, 17.219, 18.162, 21.814, 22.260, 23.087, 23.351, 24.518, 25.456, 26.846, 28.376, 29.648, 30.509, 31.226, and 32.328. Form Ib also can be characterized by having a specific X-ray powder diffraction pattern graph as illustrated herein.

The polymorph Form Ib also can be characterized by having an FTIR spectrum with one or more of the approximate FTIR peaks selected from the group consisting of 3334, 3194, 1597, 1556, 1492, 1446, 1400, 1367, 1338, 1314, 1294, 1255, 1190, 1075, 1020, 992, 928, 835, 797, 748, and 733. Further, Form Ib can be characterized by having a specific FTIR graph as illustrated herein.

In another embodiment, the invention provides a crystalline polymorph of the compound of Formula (1), wherein the polymorph is designated as polymorph Form II. The polymorph Form II can be characterized by having one or more of the approximate X-ray powder diffraction interplanar spacing peaks selected from the group consisting of 4.794, 7.020, 7.747, 8.104, 9.457, 11.483, 13.223, 15.010, 15.693, 16.943, 18.222, 19.552, 22.498, 23.003, and 29.490. Form II also can be characterized by having a specific X-ray powder diffraction pattern graph as illustrated herein.

The polymorph Form II also can be characterized by having an FTIR spectrum with one or more of the approximate FTIR peaks selected from the group consisting of 332, 3207, 1555, 1499, 1443, 1396, 1289, 1188, 1154, 1083, 1018, 940, 835, and 796. Further, Form II can be characterized by having a specific FTIR graph as illustrated herein.

The polymorph Forms also can be described in relation to differential scanning calorimetry (DSC) characteristics. For example, the polymorphic Forms can have an endothermic maximum at about 310° C. when measured using DSC. In specific embodiments, polymorph Form Ib can have a DSC curve exhibiting peaks at about 92.7° C., 120.1° C., and 125.9° C., and polymorph Form II can have a DSC curve exhibiting peaks at about 68.0° C., 95.3° C., 115.8° C., and 126.3° C.

In certain embodiments, the various polymorphs of the invention may be provided in the form of a hydrate. For example, the crystalline polymorphs may have a water content of about 10% to about 40% by weight. In specific embodiments, the hydrate can be characterized as being stable for a length of time when stored at a temperature of about 25° C. and a relative humidity of about 60%. Preferably, stability is evidenced by an absence of any significant additional water uptake by the hydrate and/or the absence of any significant water loss by the hydrate.

In another aspect, the invention is also directed to methods of preparing the crystalline polymorphic forms described herein. In some embodiments, the method can comprise forming a solution of the compound according to Formula (1) in a suitable polar solvent, such as an alcohol (e.g., methanol) and water, optionally with heating, and further optionally with mixing. When heating is used, it can be useful to cool the solution, such as to ambient conditions, to facilitate precipitation of the polymorph. Specific anti-solvents can be used to precipitate the desired polymorph from the solution, such anti-solvents preferably being non-polar solvents (e.g., methyl isobutylketone and tetrahydrofuran). In certain embodiments, the method can comprise isolating impurities from the compound of Formula (1). This can include combining the compound with a material suitable for absorbing and/or adsorbing contaminants (e.g., materials other than the actual compound of Formula (1)). Specifically, the material can comprise activated carbon.

In still another aspect, the invention is directed to pharmaceutical compositions. In one embodiment, the invention provides a pharmaceutical composition comprising: a therapeutically effective amount of the crystalline polymorph Form Ia or a pharmaceutically acceptable prodrug or pharmaceutically active metabolite thereof and a pharmaceutically acceptable carrier. In another embodiment, the invention provides a pharmaceutical composition comprising: a therapeutically effective amount of the crystalline polymorph Form Ib or a pharmaceutically acceptable prodrug or pharmaceutically active metabolite thereof and a pharmaceutically acceptable carrier therefor. In a further embodiment, the invention provides a pharmaceutical composition comprising: a therapeutically effective amount of the crystalline polymorph Form II or a pharmaceutically acceptable prodrug or pharmaceutically active metabolite thereof; and a pharmaceutically acceptable carrier therefor.

In yet another aspect, the present invention is directed to methods of treating various conditions by administering to a subject in need of treatment a polymorph as described herein. In one embodiment, the invention provides a method for treating a condition selected from the group consisting of abnormal cell proliferation, inflammation, asthma, and arthritis, said method comprising administering to a subject in need of treatment a therapeutically effective amount of the crystalline polymorph, Form Ia or a pharmaceutically acceptable prodrug or pharmaceutically active metabolite thereof In another embodiment, the invention provides a method for treating a condition selected from the group consisting of abnormal cell proliferation, inflammation, asthma, and arthritis, said method comprising administering to a subject in need of treatment a therapeutically effective amount of the crystalline polymorph, Form Ib or a pharmaceutically acceptable prodrug or pharmaceutically active metabolite thereof. In a further embodiment, the invention provides a method for treating a condition selected from the group consisting of abnormal cell proliferation, inflammation, asthma, and arthritis, said method comprising administering to a subject in need of treatment a therapeutically effective amount of the crystalline polymorph, Form II or a pharmaceutically acceptable prodrug or pharmaceutically active metabolite thereof.

In specific embodiments, the polymorph compound of the invention may be combined with further polymorphs as described herein. In other embodiments, the polymorph compound may be combined with one or more further active agents (e.g., methotrexate).

### BRIEF DESCRIPTION OF THE DRAWINGS

Having thus described the invention in the foregoing general terms, reference will now be made to the accompanying drawings, which are not necessarily drawn to scale, and wherein:

FIG. 1 is an X-ray powder diffraction pattern graph of a polymorph, designated Form Ia, according to one embodiment of the invention, wherein the top line is the actual XRD pattern and the bottom line illustrates the relative intensities of the various peaks;

FIG. 2 is an X-ray powder diffraction pattern graph of a polymorph, designated Form Ib, according to one embodiment of the invention, wherein the top line is the actual XRD pattern and the bottom line illustrates the relative intensities of the various peaks; and

FIG. 3 is a differential scanning calorimetry curve for polymorph Form Ib according to one embodiment of the invention;

FIG. 4 is a curve obtained by thermogravimetric analysis of the polymorph of Form Ib according to one embodiment of the invention;

FIG. 5 is a curve obtained by dynamic vapor sorption analysis of the polymorph of Form Ib according to one embodiment of the invention showing mass change versus time while cycling relative humidity (RH);

FIG. 6 is a curve obtained by dynamic vapor sorption analysis of the polymorph of Form Ib according to one embodiment of the invention showing mass change as a function of RH;

FIG. 7 is a curve obtained by dynamic vapor sorption analysis of the polymorph of Form Ib according to one embodiment of the invention showing water content versus time while cycling RH;

FIG. 8 is a curve obtained by dynamic vapor sorption analysis of the polymorph of Form Ib according to one embodiment of the invention showing water content as a function of RH;

FIG. 9 is a FTIR spectrum of polymorph Form Ib according to one embodiment of the invention;

FIG. 10 is an X-ray powder diffraction pattern graph of a polymorph, designated Form II, according to one embodiment of the invention, wherein the top line is the actual XRD pattern and the bottom line illustrates the relative intensities of the various peaks;

FIG. 11 is a differential scanning calorimetry curve for polymorph Form II according to one embodiment of the invention;

FIG. 12 is a curve obtained by thermogravimetric analysis of the polymorph of Form II according to one embodiment of the invention;

FIG. 13 is a curve obtained by dynamic vapor sorption analysis of the polymorph of Form II according to one embodiment of the invention showing mass change versus time while cycling RH;

FIG. 14 is a curve obtained by dynamic vapor sorption analysis of the polymorph of Form II according to one embodiment of the invention showing mass change as a function of RH;

FIG. 15 is a curve obtained by dynamic vapor sorption analysis of the polymorph of Form II according to one embodiment of the invention showing water content versus time while cycling RH;

FIG. 16 is a curve obtained by dynamic vapor sorption analysis of the polymorph of Form II according to one embodiment of the invention showing water content as a function of RH; and

FIG. 17 is a FTIR spectrum of polymorph Form II according to one embodiment of the invention.

### DETAILED DESCRIPTION

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

### I. Definitions

The term “polymorph” as used herein means a crystalline form of a compound with a distinct spatial lattice arrangement as compared to other crystalline forms of the same compound. A polymorph may also be defined as different unsolvated crystal forms of a compound. Specific polymorphs can have different chemical and physical properties. Unless otherwise specifically noted, the term polymorph is intended to encompass pseudopolymorphs as well.

The term “pseudopolymorph” as used herein means a polymorph that incorporates a solvent or water into the crystal lattice of the compound. Specific examples of pseudopolymorphs encompassed by the invention include solvates (which are understood to be crystalline forms incorporating solvent into the crystal lattice of the compound) and hydrates (which are understood to mean crystalline forms incorporating water in the crystal lattice of the compound). A pseudopolymorph may also be a desolvated solvate (i.e., a form that can be made by removing the solvent from a solvate) or a dehydrated hydrate (i.e., a form that can be made by removing the water from a hydrate).

The term “prodrug” as used herein means a compound that may be converted under physiological conditions or by solvolysis to the specified compound.

The term “active metabolite” as used herein means a physiologically active compound which results from the metabolism of a compound of the invention, or a prodrug thereof, when such compound or prodrug is administered to a mammal. Metabolites of a compound may be identified using routine techniques known in the art.

The terms “therapeutically effective amount” or “therapeutically effective dose” as used herein are interchangeable and mean a concentration of a compound according to the invention, or a biologically active variant thereof, sufficient to elicit the desired therapeutic effect according to the methods of treatment described herein.

The term “pharmaceutically acceptable carrier” as used herein means any material that is conventionally used in the art to facilitate the storage, administration, and/or the healing effect of a biologically active agent, and such term is intended to encompass any further terms for materials providing the same function, such as “diluent,” “vehicle,” “adjuvant,” and the like.

The term “intermittent administration” as used herein means administration of a therapeutically effective dose of a composition according to the invention, followed by a time period of discontinuance, which is then followed by another administration of a therapeutically effective dose, and so forth.

The term “antiproliferative agent” as used herein means a compound that decreases the hyperproliferation of cells.

The term “abnormal cell proliferation” as used herein means a disease or condition characterized by the inappropriate growth or multiplication of one or more cell types relative to the growth of that cell type or types in an individual not suffering from that disease or condition.

The term “cancer” as used herein means a disease or condition characterized by uncontrolled, abnormal growth of cells, which can spread locally or through the bloodstream and lymphatic system to other parts of the body. The term includes tumor-forming or non-tumor forming cancers, and includes various types of cancers, such as primary tumors and tumor metastasis.

The term “tumor” as used herein means an abnormal mass of cells within a multicellular organism that results from excessive cell division that is uncontrolled and progressive, also called a neoplasm. A tumor may either be benign or malignant.

The term “fibrotic disorders” as used herein means fibrosis and other medical complications of fibrosis which result in whole or in part from the proliferation of fibroblasts.

The term “arthritis” as used herein means an inflammatory disorder affecting joints that can be infective, autoimmune, or traumatic in origin.

### II. Compounds

The present invention provides multiple polymorphic crystalline forms of the compound according to Formula (1), (S)-2-{4-[2-(2,4-diamino-quinazolin-6-yl)-ethyl]-benzoylamino}-4-methylene-pentanedioic acid, dipotassium salt. The invention also provides pseudopolymorphs of the compound according to Formula (1), particularly hydrates of the compound.

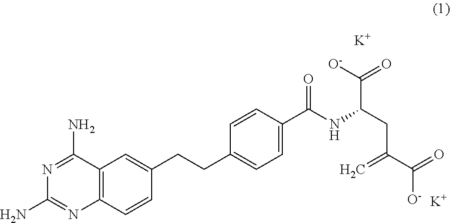

The compound of Formula (1) is a metabolically inert antifolate. As recognized in the art, antifolates are compounds that interfere with various stages of folate metabolism. Thus, the polymorphs of the invention can be used in pharmaceutical compositions useful for the treatment of diseases and conditions related to or capable of being treated by disruption of folate metabolism, or other biological mechanisms related to folate metabolism.

In certain embodiments, the present invention can encompass pure, single polymorphs as well as mixtures comprising two or more different polymorphs. A pure, single polymorph may be substantially free from other polymorphs. Substantially free means that other polymorph(s) are present in an amount less than about 15% by weight, less than about 10% by weight, less than about 5% by weight, or less than about 1% by weight. Someone with ordinary skill in the art would understand the phrase “in an amount less than about 15% by weight” to mean that the polymorph of interest is present in a polymorph mixture in an amount more than about 85% by weight. Likewise, the phrase “less than about 10% by weight” would mean that the polymorph of interest is present in a polymorph mixture in an amount more than about 90% by weight, and so forth. In other embodiments, a pure single polymorph may be completely free from other polymorphs.

In other embodiments, the polymorph forms of the invention can be described as being substantially pure, meaning that each polymorph form of the compound of Formula (1) is isolated at a purity of at least about 90% by weight (i.e., less than about 10% by weight impurities, including other polymorph forms of the compound), at a purity of at least about 95% by weight, at least about 98% by weight, or at least about 99% by weight.

Polymorphs of the compounds of the preferred embodiments of the present invention may be desirable because a particular polymorph of a compound may have better physical and chemical properties than other polymorphic forms of the same compound. For example, one polymorph may have increased solubility in certain solvents. Such added solubility may facilitate formulation or administration of the compounds of the preferred embodiments of the present invention. Different polymorphs may also have different mechanical properties (e.g., different compressibility, compatibility, and ability to be specifically formed, such as in a tablet), which may influence performance of the drug, and thus influence formulation of the drug. A particular polymorph may also exhibit a different dissolution rate in the same solvent, relative to another polymorph. Different polymorphs may also have different physical stability (e.g., solid-state conversion from metastable polymorph to a more stable polymorph) and/or different chemical stability (e.g., reactivity).

In certain embodiments, individual crystalline forms of the compound of Formula (1) may be identified by a characteristic X-ray powder diffraction pattern. X-ray powder diffraction is a known technique using X-ray radiation on a sample (e.g., a powder or microcrystalline material) for structural characterization of the sample. Powder diffraction data are usually presented as a diffractogram in which the diffracted intensity I is shown as a function either of the scattering angle 2θ or as a function of the scattering vector q. Identification is performed by comparison of the diffraction pattern to a known standard or to a database such as the International Centre for Diffraction Data's Powder Diffraction File (PDF) or the Cambridge Structural Database (CSD). The fundamental physics upon which the technique is based provides high precision and accuracy in the measurement of interplanar spacings, typically to fractions of an Angstrom, resulting in authoritative identification of specific materials, even distinct polymorphic forms of the same crystalline compound, each of which produces a distinctive diffraction pattern. Both the positions (corresponding to lattice spacings) and the relative intensity of the lines are indicative of a particular phase and material. Equipment useful for measuring such data is known in the art, such as a Shimadzu XRD-6000 X-ray diffractometer, and any such equipment can be used to measure the compounds according to the present invention. In certain embodiments, XRD analysis can be carried out using a device incorporating a copper (Cu) anode providing kappa alpha (Kα) radiation. Thus, specific polymorphs according to the invention may be identified and described in relation to the representative graph and/or the approximate X-ray powder diffraction “d-spacing” peaks (i.e., interplanar spacing peaks at 2°θ) obtained in XRD analysis, particularly using Cu Kα radiation.

In further embodiments, individual crystalline forms of the compound of Formula (1) may be identified by the characteristic curves obtained by differential scanning calorimetry (DSC), wherein the difference in the amount of heat required to increase the temperature of a sample and reference are measured as a function of temperature. The result of a DSC analysis particularly can be a curve of heat flux versus temperature change. By observing the difference in heat flow between the sample and reference, DSC analysis can measure the amount of heat absorbed or released during phase transitions and can also identify more subtle phase changes, such as glass transition. DSC is widely used in industrial settings as a quality control instrument due to its applicability in evaluating sample purity. Equipment useful for measuring such data is known in the art, such as a PerkinElmer DSC 7 Differential Scanning calorimeter, and any such equipment can be used to measure the compounds according to the present invention. In certain embodiments, DSC analysis can be carried out by heating over a specific temperature range at a specific heating rate. The analysis may further be described in terms of the sample holder, such as a closed gold pan. Thus, specific polymorphs according to the invention may be identified and described in relation to the representative graph and/or the approximate peaks obtained in DSC analysis, particularly heating at a rate of about 10° C. per minute.

In other embodiments, individual crystalline forms of the compound of Formula (1) may be identified by the characteristic curves obtained by thermogravimetric analysis (TGA), which can be used to describe weight as a function of temperature. TGA analysis relies on a high degree of precision in measurement of weight, temperature, and temperature change. The analyzer usually consists of a high-precision balance with a pan (generally platinum) loaded with the sample. The pan is placed in a small electrically heated oven with a thermocouple to accurately measure the temperature. The atmosphere may be purged with an inert gas to prevent oxidation or other undesired reactions. Analysis is carried out by raising the temperature gradually and plotting weight against temperature. Equipment useful for measuring such data is known in the art, such as an Orton TG730, and any such equipment can be used to measure the compounds according to the present invention. In certain embodiments, TGA analysis can be carried out by heating over a specific temperature range at a specific heating rate. Thus, specific polymorphs according to the invention may be identified and described in relation to the representative graph and/or the approximate peaks obtained in TGA analysis, particularly heating at a rate of about 10° C. per minute.

In additional embodiments, individual crystalline forms of the compound of Formula (1) may be identified by the characteristic curves obtained by dynamic vapor sorption (DVS), wherein a sample is subjected to varying conditions of humidity and temperature, and the response of the sample is measured gravimetrically. The result of a DVS analysis particularly can be a dual curve providing sample weight percent as a function of relative humidity (RH) over time, a dual curve providing sample water content as a function of RH over time, a curve providing weight percent in relation to RH, or a curve providing water content in relation to RH. Equipment useful for measuring such data is known in the art, such as a TA Instruments VTI-SA3, and any such equipment can be used to measure the compounds according to the present invention. In certain embodiments, DVS analysis can be carried out by scanning at a series of specific RH values. Thus, specific polymorphs according to the invention may be identified and described in relation to the representative graph and/or the approximate peaks obtained in DVS analysis, particularly scanning at 0%, 50%, and 95% RH with a 5% RH per hour change and a hold time of 5 hours at 0% and 95% RH.

In still further embodiments, individual crystalline forms of the compound of Formula (1) may be identified by the characteristic curves obtained by infrared (IR) spectrum analysis. Equipment useful for measuring such data is known in the art, such as a PerkinElmer Spectrum BX Fourier transform infrared (FTIR) spectrometer, and any such equipment can be used to measure the compounds according to the present invention. Thus, specific polymorphs according to the invention may be identified and described in relation to the representative graph and/or the approximate peaks obtained in FTIR analysis.

The present invention also provides hydrate forms of the compound of Formula (1). In certain embodiments, a hydrate of the compound of Formula (1) can comprise about 10% to about 40% by weight water. In other embodiments, a hydrate of the compound of Formula (1) can comprise water in an amount of about 10% to about 35% by weight, about 10% to about 30% by weight, about 12% to about 28% by weight, or about 14% to about 28% by weight. A hydrate form according to the present invention preferably is stable when stored at a temperature of about 25° C. (+/−2° C.) and a relative humidity of about 60% (+/−5% RH). Preferably, such stability can be characterized by the absence of any significant additional water uptake by the hydrate (e.g., an uptake of less than about 5%, less than about 4%, less than about 3%, less than about 2%, or less than about 1%). Such stability further may be characterized by the absence of any significant water loss by the hydrate (e.g., a loss of no more than about 5%, no more than about 4%, no more than about 3%, no more than about 2%, or no more than about 1%). The hydrate preferably is stable for a storage time under the above conditions of at least about 1 month, at least about 2 months, at least about 3 months, at least about 6 months, at least about 9 months, or at least about 12 months.

Individual polymorphic forms of the compound of Formula (1) can be obtained though appropriate solvent and anti-solvent combination systems. The terms “solvent” and “anti-solvent” are not intended to limit the scope of materials that may be used but are rather used to describe the relationship between the materials used in that they have distinctly opposite properties. The “solvent” particularly can be a polar liquid, which may be referred to as a polar solvent. The “anti-solvent” particularly can be a non-polar liquid, which may be referred to as a non-polar solvent.

A sample of the compound of Formula (1) may be solubilized using any suitable polar solvent(s). Non-limiting examples of polar solvents that may be used include water, methanol, ethanol, n-propanol, isopropanol, n-butanol, dimethylformamide (DMF), dimethyl sulfoxide (DMSO), and combinations thereof. Of course, other polar solvents that would be recognizable by one of skill in the art in light of the present disclosure also could be used. For example, in certain embodiments, a suitable polar solvent may be any material that is liquid at ambient conditions (e.g., approximately 18-25° C. and 1 atm) and has a dielectric constant of at least about 15. In specific embodiments, the polar solvent can be a mixture of water and at least one further polar solvent, particularly an alcohol, and more particularly methanol.

The solution of the compound of Formula (1) in a polar solvent system can be used directly with an anti-solvent to isolate the desired polymorphic form. In some embodiments, the solubilized compound of Formula (1) may be treated to improve compound purity. For example, a quantity of activated carbon may be added to the solution to form a mixture. Of course, any further material useful to remove impurities from a solution also could be used. The mixture with the purity-increasing agent (i.e., activated carbon) is preferably filtered prior to proceeding with isolation of the desired polymorphic form. For example, the mixture can be filtered through a SEITZ® K200 filter, and/or a CELITE® filter pad (e.g., approximately 5 mm thickness), and/or any further filter media suitable for removing the activated carbon or other purity-increasing agent from the solution. The recovered, purified solution can then proceed to treatment with the anti-solvent.

The solution of the compound of Formula (1) in the polar solvent can be slurried by adding thereto a suitable non-polar solvent. Non-limiting examples of non-polar solvents that may be used include tetrahydrofuran (THF), methyl ethyl ketone (MEK), methyl isobutyl ketone (MIBK), hexane, benzene, toluene, diethyl ether, chloroform, methyl acetate, ethyl acetate, dioxane, and combinations thereof. Of course, other non-polar solvents that would be recognizable by one of skill in the art in light of the present disclosure also could be used. For example, in certain embodiments, a suitable non-polar solvent may be any material that is liquid at ambient conditions and has a dielectric constant of less than about 15.

The choice of non-polar solvent may be particularly related to the polymorphic form that is recovered. Examples of such specificity are provided herein. The slurry formed as described above can be allowed to sit, preferably with stirring, for a defined time period to allow maximum formation of solid product. The solid product may be recovered by filtering and optional washing. In specific embodiments, the filtered product can be washed first with a mixture of a polar solvent and a non-polar solvent and washed a second time with a non-polar solvent alone.

The Examples appended hereto describe testing whereby different solvent/anti-solvent systems may be evaluated to identify systems useful for formation of stable, crystalline salts. Provided below are two specific stable, crystalline salt polymorphic forms of the compound of Formula (1) that have been identified.

A. Polymorph Form I

Polymorph Form I can be prepared as described herein, wherein the solvent is a mixture of water and methanol and the anti-solvent is MEK or MIBK. More specifically, the compound of Formula (1) can be solubilized in the mixture of water and methanol, and the resulting solution can be slurried in MEK or MIBK. The slurry may be heated initially to a temperature of about 40° C. and allowed to cool to ambient temperature. Polymorph Form I is stable at 25° C. (+/−2° C.) and 60% relative humidity (+/−5% RH) with no significant water uptake.

The X-ray powder diffraction pattern for Form I was obtained using a PANALYTICAL® X'Pert Diffractometer (PANalytical B.V., The Netherlands) with 40 kV/40 mA Cu anode. A 0.4 mm thick stationary sample was used and was covered with KAPTON® foil. A measurement range of 2-50° Theta was used with a step size of 0.02° and a step time of 2.4 seconds.

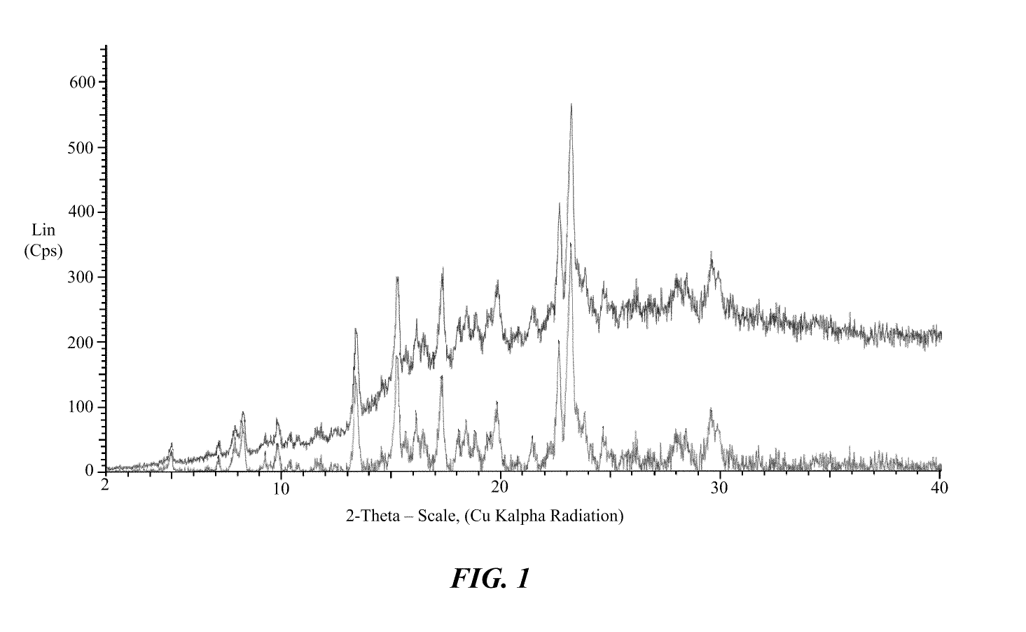

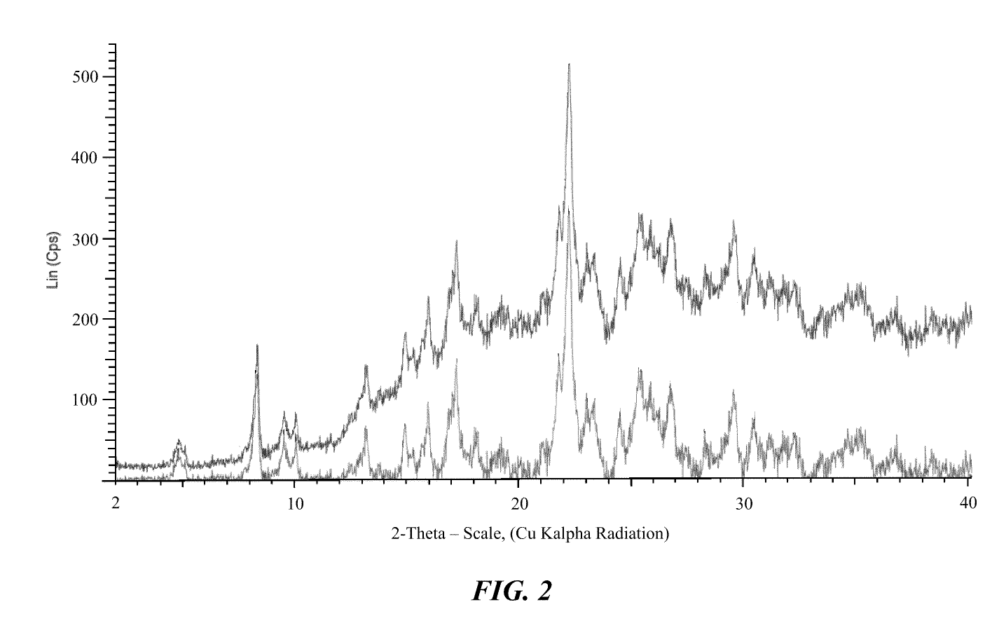

The X-ray powder diffraction pattern for Form I using MEK as the anti-solvent is illustrated in FIG. 1. The X-ray powder diffraction pattern for Form I using MiBK as the anti-solvent is illustrated in FIG. 2. As seen therein, the XRD pattern for Form I exhibits a consistent series of main peaks; however, Form I can vary slightly depending upon the anti-solvent employed to isolate the polymorph. Thus, polymorphic Form I according to the invention may be described as having two sub-forms—i.e., polymorphic Form Ia (MEK anti-solvent) and Form Ib (MIBK anti-solvent).

Polymorphic Form Ia has the diffractogram shown in FIG. 1 and is obtained using water/methanol as the solvent and MEK as the anti-solvent. Form Ia can be characterized by the approximate X-ray powder diffraction “d-spacing” peaks (i.e., interplanar spacing peaks at 2°θ) provided below in Table 1. In some embodiments, the exact interplanar spacing peaks provided below may exhibit a variation (i.e., plus or minus) of up to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or 0.2 degrees.

TABLE 1

| Angle 2θ° | Intensity % |
|---|---|
| 4.946 | 7 |
| 7.118 | 6 |
| 7.875 | 14 |
| 8.238 | 20 |
| 9.229 | 7 |
| 9.822 | 11 |
| 13.400 | 42 |
| 15.271 | 50 |
| 15.658 | 17 |
| 16.128 | 26 |
| 16.459 | 18 |
| 17.286 | 41 |
| 18.088 | 17 |
| 18.452 | 22 |
| 18.889 | 17 |
| 19.490 | 14 |
| 19.837 | 30 |
| 21.456 | 14 |
| 22.658 | 57 |
| 23.168 | 100 |
| 23.811 | 26 |
| 24.691 | 19 |
| 28.436 | 17 |
| 29.609 | 26 |

Polymorphic Form Ib has the diffractogram shown in FIG. 2 and is obtained using water/methanol as the solvent and MIBK as the anti-solvent. Form Ib can be characterized by the approximate X-ray powder diffraction “d-spacing” peaks (i.e., interplanar spacing peaks at 2°θ) provided below in Table 2. In some embodiments, the exact interplanar spacing peaks provided below may exhibit a variation (i.e., plus or minus) of up to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or 0.2 degrees.

TABLE 2

| Angle 2θ° | Intensity % |
|---|---|
| 4.811 | 10 |
| 8.316 | 37 |
| 9.542 | 17 |
| 10.047 | 14 |
| 13.189 | 19 |
| 14.946 | 20 |
| 15.973 | 28 |
| 17.219 | 43 |
| 18.162 | 16 |
| 21.814 | 46 |
| 22.260 | 100 |
| 23.087 | 28 |
| 23.351 | 28 |
| 24.518 | 23 |
| 25.456 | 40 |
| 26.846 | 33 |
| 28.376 | 17 |
| 29.648 | 31 |
| 30.509 | 21 |
| 31.226 | 15 |
| 32.328 | 16 |

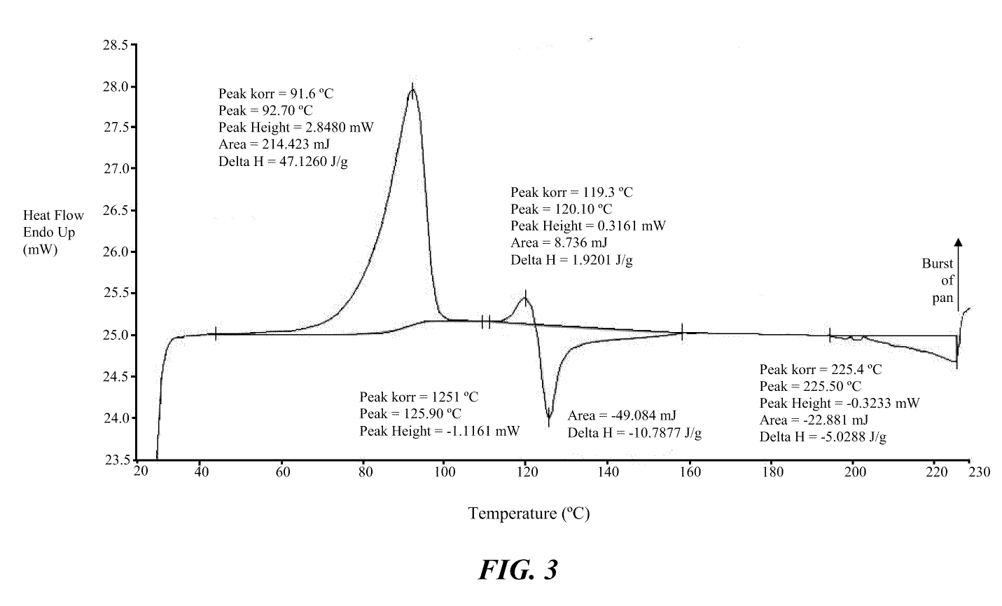

Form I is further characterized by having an endothermic maximum of about 310° C., as determined using differential scanning calorimetry (DSC). In particular, the crystalline solid decomposes above this maximum In specific embodiments, polymorphic Form Ib can be characterized by the DSC curve shown in FIG. 3, which was obtained using a PerkinElmer calorimeter wherein the sample was provided in a closed gold pan heating from 25° C. to 230° C. at a rate of about 10° C. per minute. Specifically, the DSC curve for Form Ib can be characterized by having peaks at 92.7° C., 120.1° C., and 125.9° C. The peak at approximately 93° C. indicated a loss of water. Although not wishing to be bound by theory, the peak at approximately 120° C. is believed to arise from a minor structural change at this temperature. In some embodiments, the exact DSC curve peaks described above may exhibit a variation (i.e., plus or minus) of up to 0.02, 0.05, 0.1, 0.2, 0.3, 0.4, or 0.5° C.

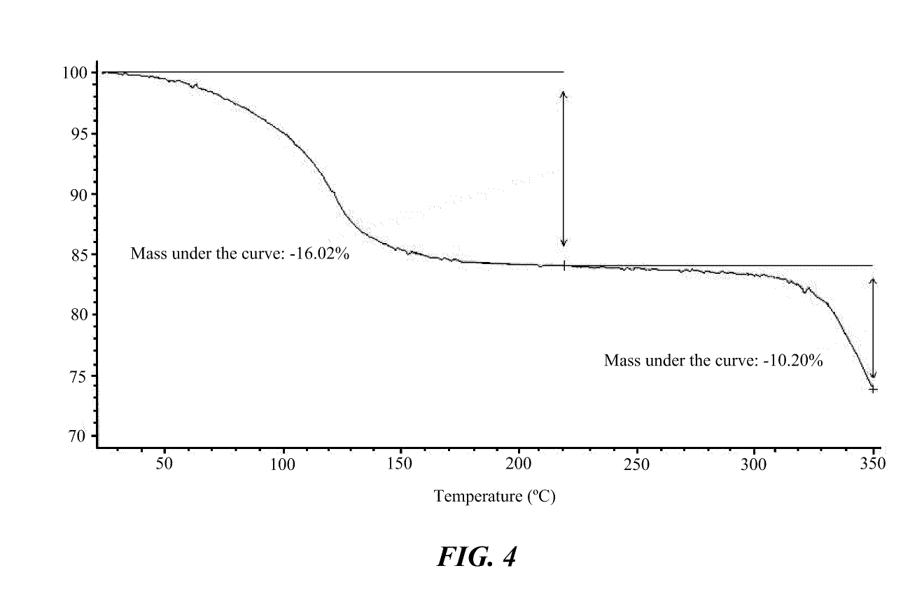

In other embodiments, polymorphic Form Ib can be characterized by the TGA curve shown in FIG. 4. The thermogravimetric analysis was carried out by heating over a temperature range of room temperature to 250° C. by heating at a rate of about 10° C. per minute.

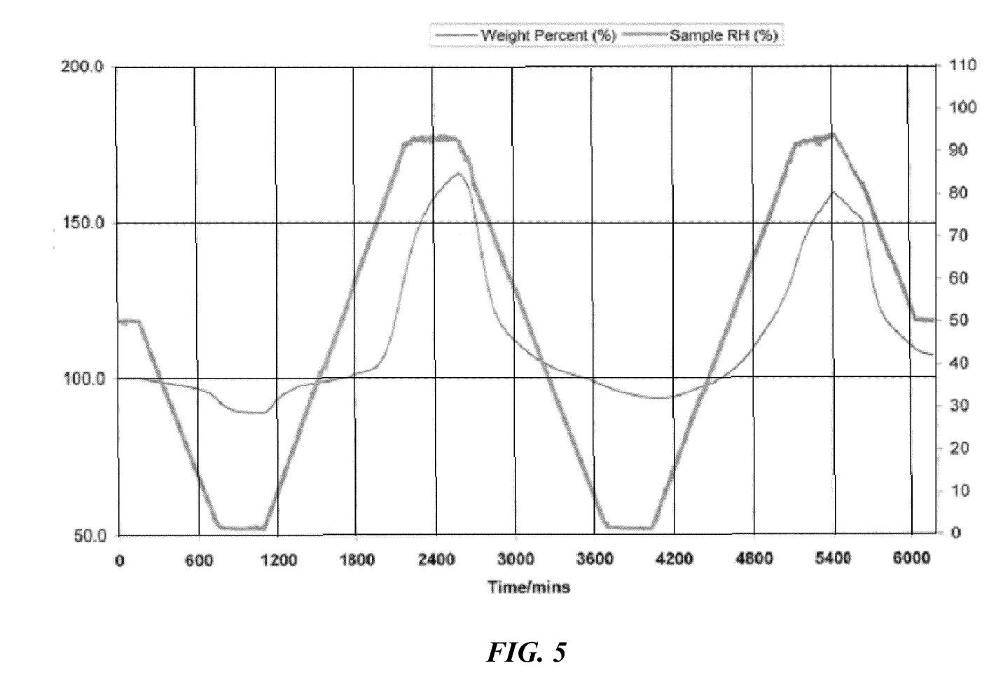

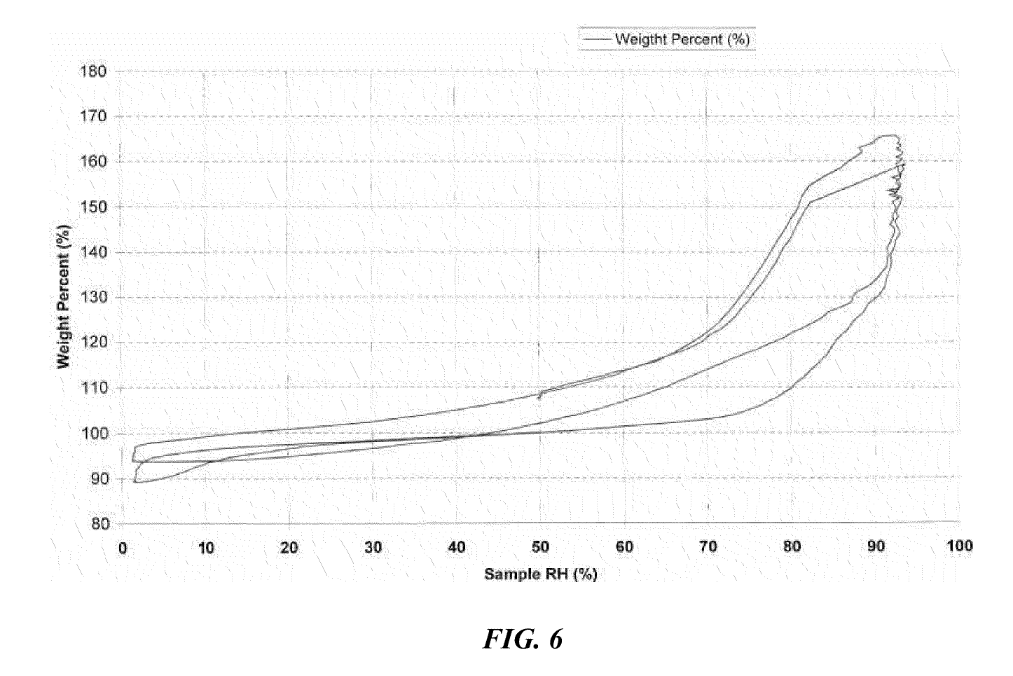

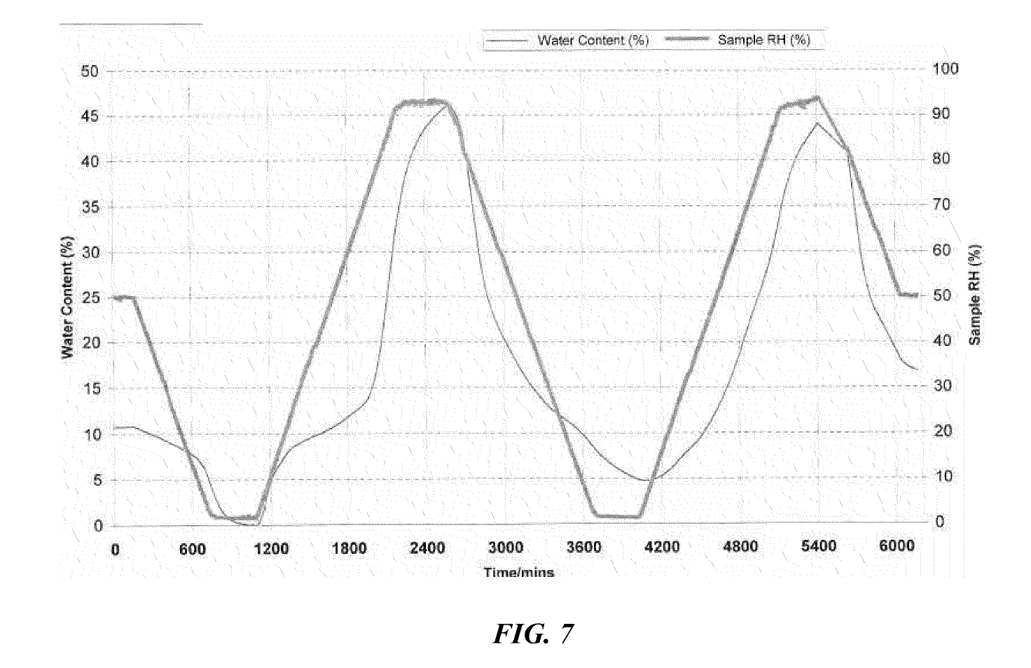

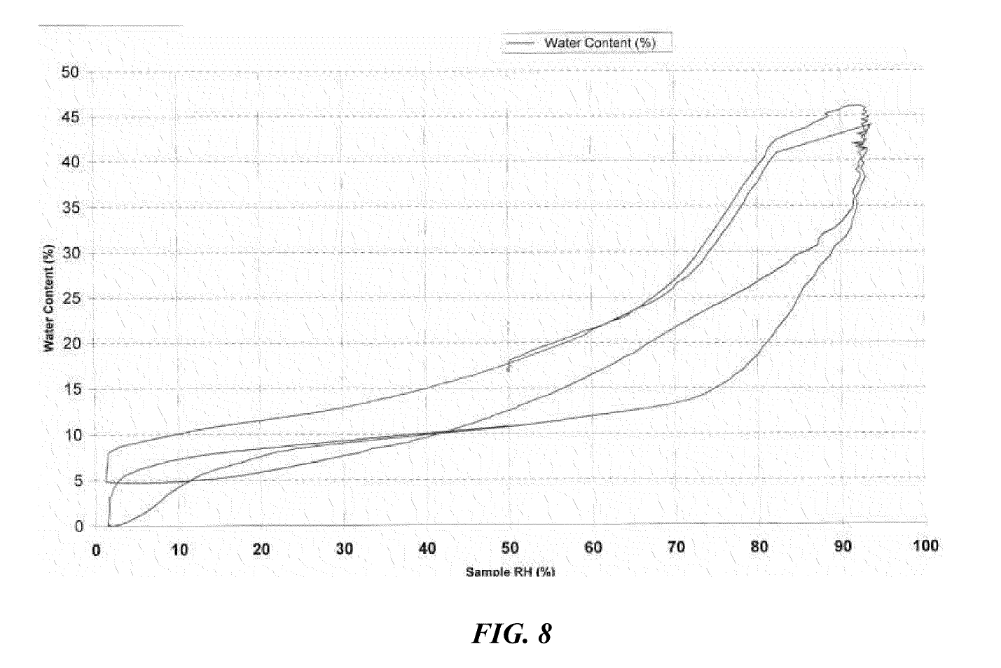

In additional embodiments, polymorphic Form Ib can be characterized by the various DVS curves shown in FIG. 5 through FIG. 8. The DVS analysis was carried out by scanning while relative humidity was cycled at 50% RH, 0% RH, 95% RH, 0% RH, 95% RH, and 50% RH at a rate of change of 5% RH with hold times of 5 hours at 0% and 95% RH.

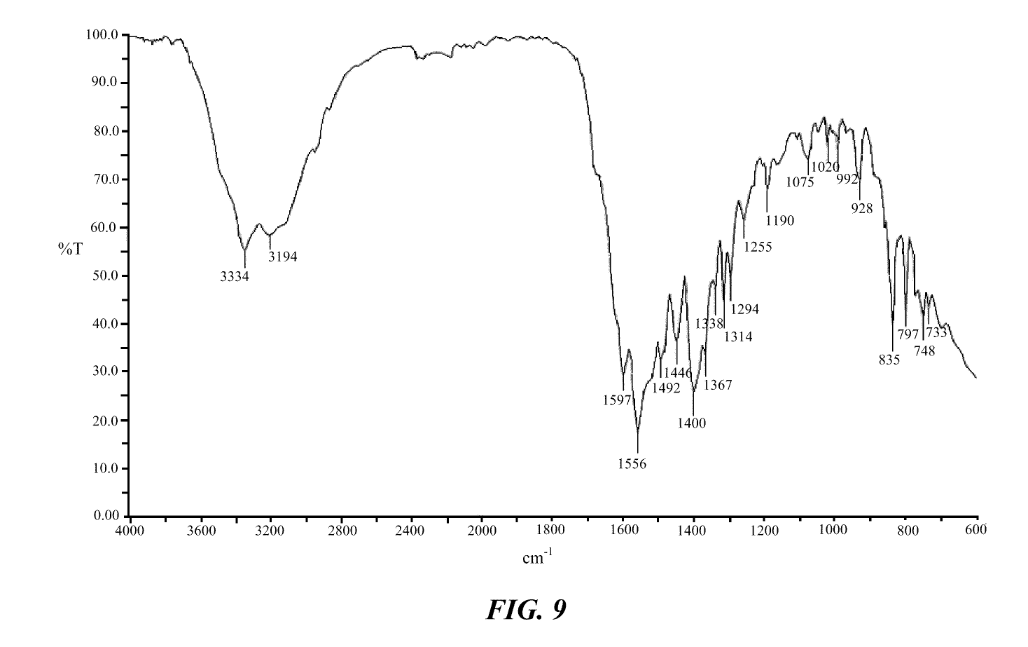

In still further embodiments, polymorphic Form Ib can be characterized by the IR spectrum shown in FIG. 9, which was obtained using a PerkinElmer Spectrum BX Fourier transform infrared (FTIR) spectrometer. The analysis specifically was carried out according to Solvias SOP A.52,S255_{—}01. Form Ib can be characterized by the approximate FTIR peaks (cm⁻¹)provided below in Table 3. In some embodiments, the exact peak values may exhibit a variation (i.e., plus or minus) of up to 0.01, 0.05, 0.1, 0.5, 1, or 2 cm⁻¹.

TABLE 3
Peak (cm⁻¹)

3334
3194
1597
1556
1492
1446
1400
1367
1338
1314
1294
1255
1190
1075
1020
992
928
835
797
748
733

B. Polymorph Form II

Polymorph Form II can be prepared as described herein, wherein the solvent is a mixture of water and methanol and the anti-solvent is THF. More specifically, the compound of Formula (1) can be solubilized in the mixture of water and methanol, and the resulting solution can be slurried in THF. The slurry may be heated initially to a temperature of about 40° C. and allowed to cool to ambient temperature. Polymorph Form II is stable at 25° C. (+/−2° C.) and 60% relative humidity (+/−5% RH) with no significant water uptake.

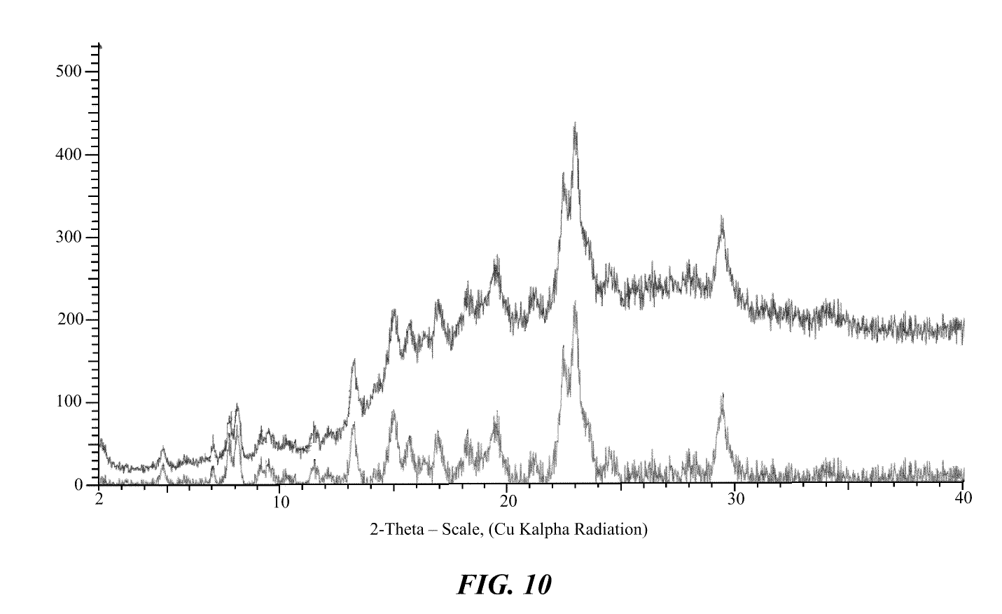

The X-ray powder diffraction pattern for Form II was obtained using the same equipment as described above in relation to Form I. The X-ray powder diffraction pattern for Form II using THF as the anti-solvent is illustrated in FIG. 10. Form II can be characterized by the approximate X-ray powder diffraction “d-spacing” peaks (i.e., interplanar spacing peaks at 2°θ) provided below in Table 4. In some embodiments, the exact interplanar spacing peaks provided below may exhibit a variation (i.e., plus or minus) of up to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or 0.2 degrees.

TABLE 4

| Angle 2θ° | Intensity % |
|---|---|
| 4.794 | 10 |
| 7.020 | 7 |
| 7.747 | 25 |
| 8.104 | 29 |
| 9.457 | 10 |
| 11.483 | 11 |
| 13.223 | 32 |
| 15.010 | 39 |
| 15.693 | 25 |
| 16.943 | 28 |
| 18.222 | 26 |
| 19.552 | 39 |
| 22.498 | 74 |
| 23.003 | 100 |
| 29.490 | 46 |

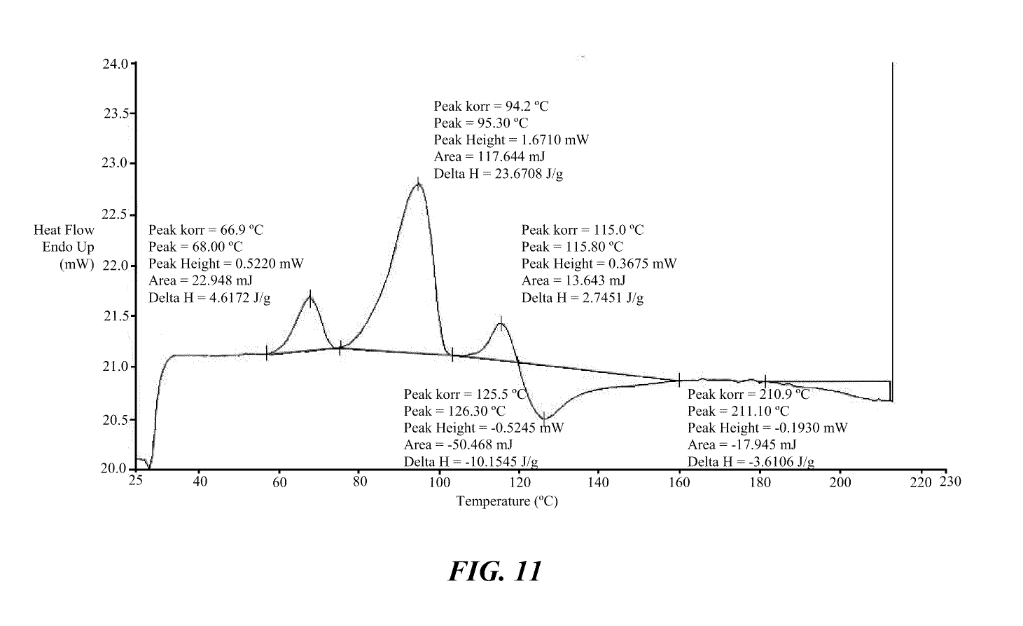

Form II is further characterized by having an endothermic maximum of about 310° C., as determined using differential scanning calorimetry (DSC). In particular, the crystalline solid decomposes above this maximum. In specific embodiments, polymorphic Form II can be characterized by the DSC curve shown in FIG. 11, which was obtained using the same equipment and test conditions as described above in relation to Form I. The peak at approximately 68° C. indicated the release of loosely bound water while the peak at approximately 95° C. indicated the release of tightly bound water. The peak at approximately 116° C. is believed to arise from a minor phase-transfer at this temperature. In some embodiments, the exact DSC curve peaks described above may exhibit a variation (i.e., plus or minus) of up to 0.02, 0.05, 0.1, 0.2, 0.3, 0.4, or 0.5° C.

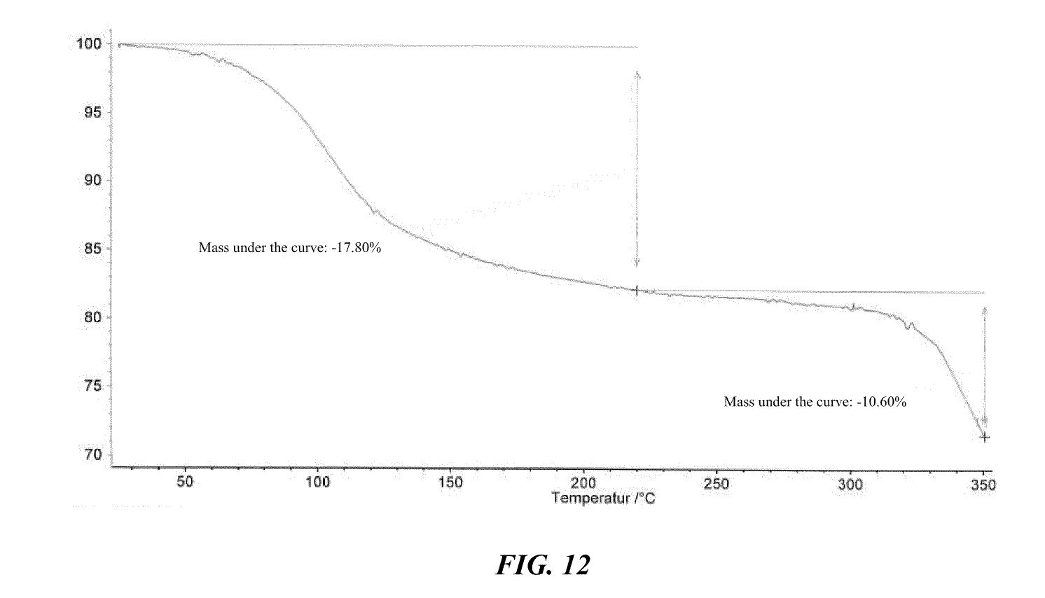

In other embodiments, polymorphic Form II can be characterized by the TGA curve shown in FIG. 12. The thermogravimetric analysis was carried out by heating over a temperature range of room temperature to 250° C. by heating at a rate of about 10° C. per minute.

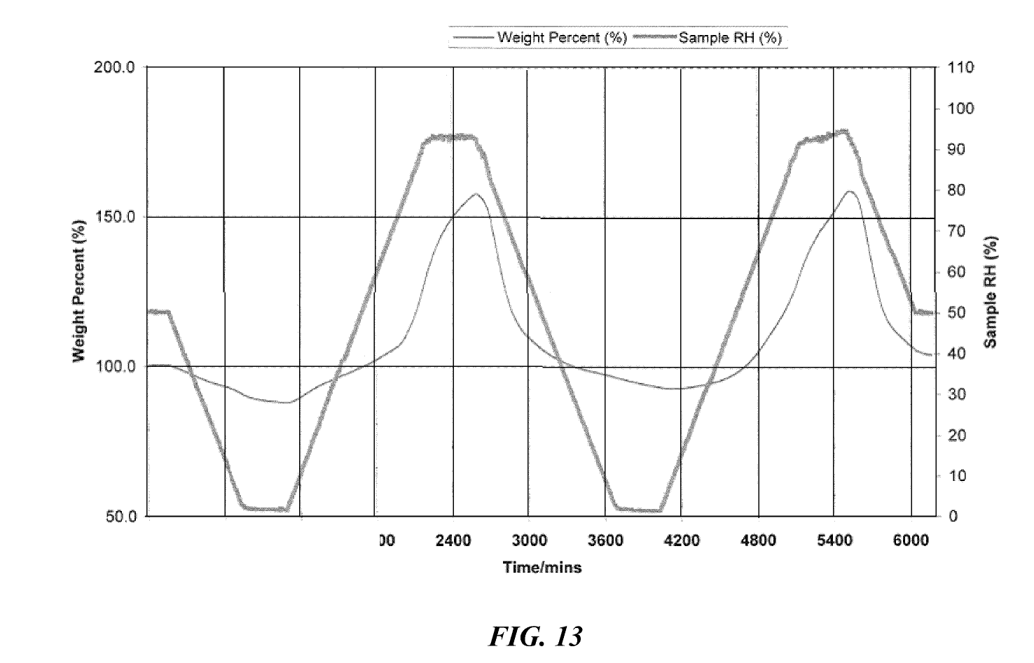

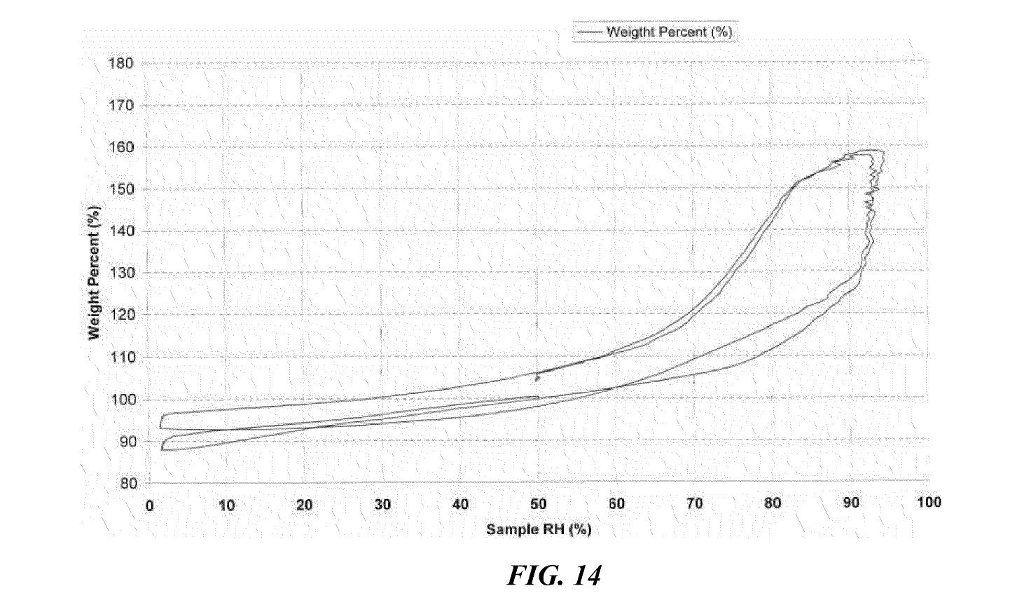

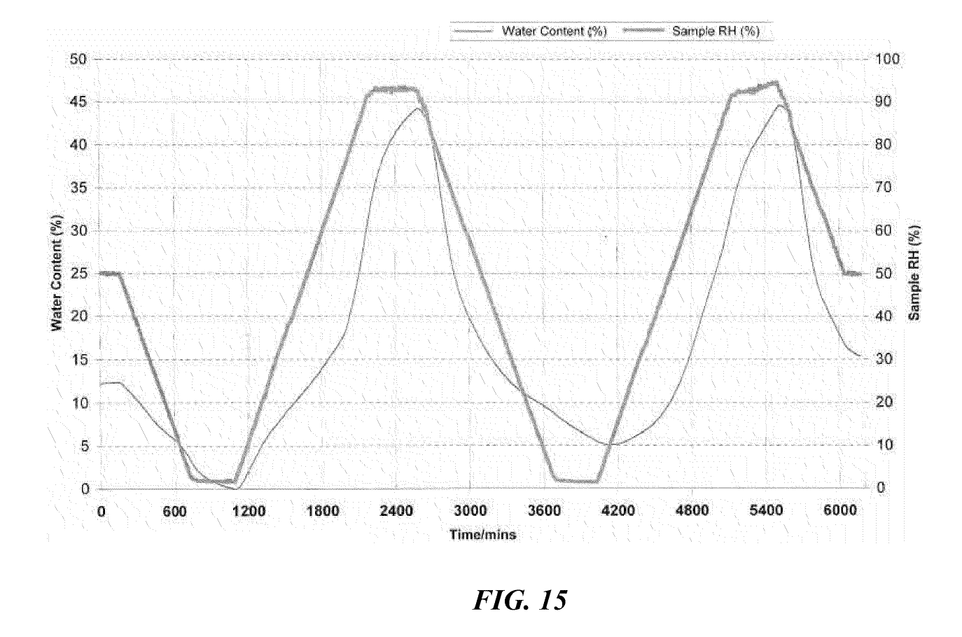

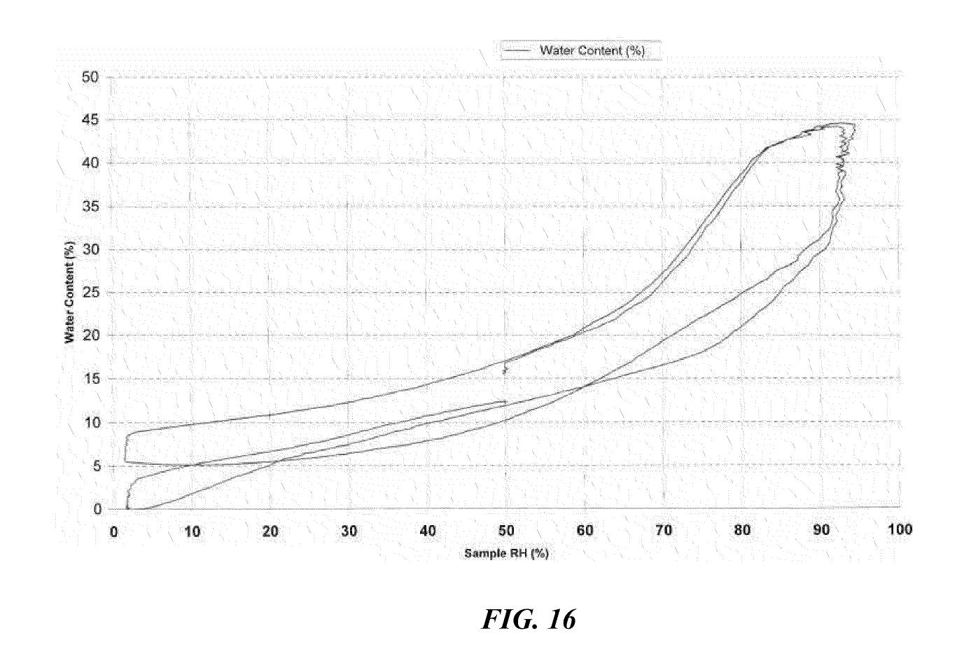

In additional embodiments, polymorphic Form II can be characterized by the various DVS curves shown in FIG. 13 through FIG. 16. The DVS analysis was carried out as described above in relation to Form I.

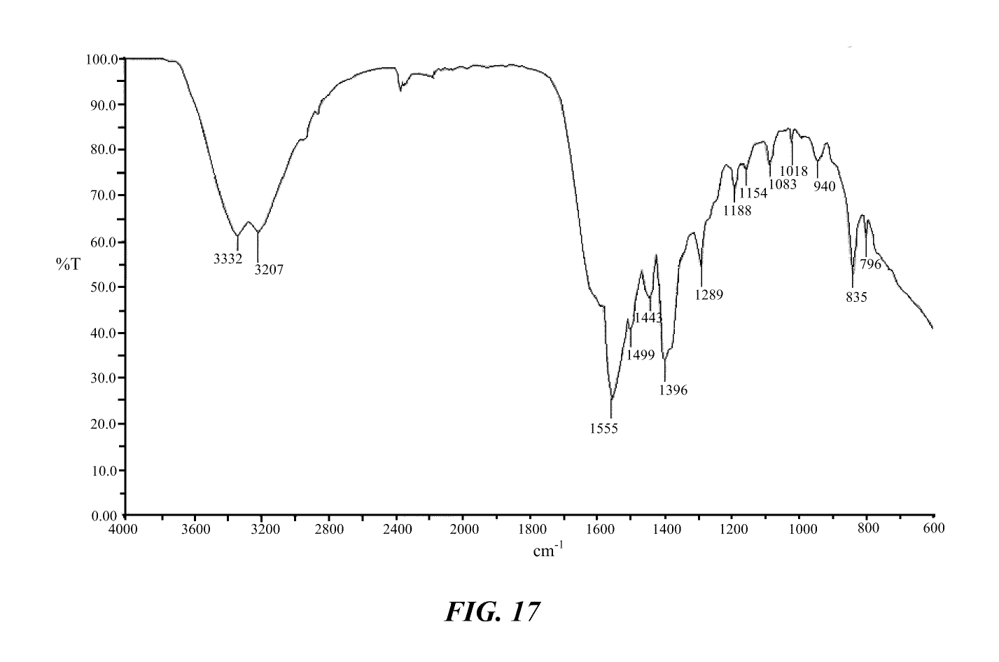

In still further embodiments, polymorphic Form Ib can be characterized by the IR spectrum shown in FIG. 17, which was obtained as described above in relation to Form I. Form II can be characterized by the approximate FTIR peaks (cm⁻¹) provided below in Table 5. In some embodiments, the exact peak values may exhibit a variation (i.e., plus or minus) of up to 0.01, 0.05, 0.1, 0.5, 1, or 2 cm⁻¹.

TABLE 5
Peak (cm⁻¹)

3332
3207
1555
1499
1443
1396
1289
1188
1154
1083
1018
940
835
796

C. Methods of Preparation

Various processes for synthesizing antifolate compounds are disclosed in U.S. Pat. No. 4,996,207, U.S. Pat. No. 5,550,128, Abraham et al. (1991) J. Med. Chem. 34:222-227, and Rosowsky et al. (1991) J. Med. Chem. 34:203-208, all of which are incorporated herein by reference. Specific methods for the synthesis of the compound according to Formula (1) are provided in U.S. Patent Application Publication No. 2009/0253719, published Oct. 8, 2009, the disclosure of which is incorporated herein by reference in its entirety.

As discussed above, in some embodiments, it may be useful to increase the purity of the compound of Formula (1) prior to forming the desired polymorphic form of the compound. Specifically, the compound of Formula (1) could be combined with a material suitable for adsorbing and/or absorbing impurities (e.g., any material that is not the actual compound of Formula (1)). For example, carbon treatment could be used. In specific embodiments, the starting compound can be mixed with activated carbon in a suitable solvent, such as a mixture of water and methanol. The mixture can then be filtered, such as through a SEITZ® K200 filter and/or a CELITE® pad. The filtered solution can be used directly in the formation of a desired polymorph according to the invention. Any further materials (e.g., zeolites) recognizable by one of skill in light of the present disclosure also are encompassed by the present disclosure as materials useful for removing impurities from the compound of Formula (1).

Formation of a desired polymorphic form can comprise heating a solution of the compound according to Formula (1) in a suitable polar solvent, such as water and/or methanol. Heating can be up to a temperature of at least about 30° C., preferably at least about 35° C., more preferably at least about 40° C. Of course, heating can be up to an even greater temperature, if desired, but heating beyond the noted temperature is not required.

The desired anti-solvent used to form the desired polymorphic form can be added during heating or after reaching the desired temperature. It may also be useful to apply agitation (e.g., mixing) during and/or after heating to the desired temperature. After heating and addition of the anti-solvent, the complete mixture can be cooled to about ambient temperature (e.g., about 18° C. to about 25° C.). Preferably, cooling is done over an extended period of time, such as passive cooling without the use of any external cooling means. Agitation may be continued during cooling. In some embodiments, it may be useful to seed the bulk solution with crystals of the desired polymorph, but this is not required. The cooled mixture should include the desired polymorph according to the invention, which can be isolated through conventional means, such as filtration.

### III. Pharmaceutical Compositions

The polymorphic forms of the compound of Formula (1) provided by the present invention may be used to form pharmaceutical compositions. The compositions also may comprise pharmaceutically acceptable prodrugs and active metabolites of the polymorphic forms. Further, the inventive compositions can be prepared and delivered by a variety of means. The pharmaceutical compositions can be prepared to deliver the polymorph together with one or more pharmaceutically acceptable carriers therefor, and optionally, other therapeutic ingredients. Carriers should be acceptable in that they are compatible with any other ingredients of the composition and not harmful to the recipient thereof. A carrier may also reduce any undesirable side effects of the agent. Non-limiting examples of carriers that could be used according to the invention are described by Wang et al. (1980) J. Parent. Drug Assn. 34(6):452-462, herein incorporated by reference in its entirety.

The pharmaceutical compositions of the invention preferably include a polymorph of the compound of Formula (1) in a therapeutically effective amount, as further described below. In certain embodiments, the amount of the polymorph in the composition is based on the overall weight of the composition. For example, in certain embodiments, the pharmaceutical composition comprises a polymorph in an amount of about 0.01 mg/g to about 100 mg/g. In further embodiments, the pharmaceutical composition comprises a polymorph in an amount of about 0.02 mg/g to about 80 mg/g, about 0.05 mg/g to about 75 mg/g, about 0.08 mg/g to about 50 mg/g, about 0.1 mg/g to about 30 mg/g, about 0.25 mg/g to about 25 mg/g, or about 0.5 mg/g to about 20 mg/g. The amount of drug can also be referenced to a unit dose (e.g., the amount of drug in a single capsule or tablet).

Compositions of the present invention may include short-term, rapid-onset, rapid-offset, controlled release, sustained release, delayed release, and pulsatile release compositions, providing the compositions achieve administration of a polymorph as described herein. See Remington's Pharmaceutical Sciences (18^{th} ed.; Mack Publishing Company, Eaton, Pa., 1990), herein incorporated by reference in its entirety. Pharmaceutical compositions according to the present invention are suitable for various modes of delivery, including oral, parenteral (including intravenous, intramuscular, subcutaneous, intradermal, intra-articular, intra-synovial, intrathecal, intra-arterial, intracardiac, subcutaneous, intraorbital, intracapsular, intraspinal, intrastemal, and transdermal), topical (including dermal, buccal, and sublingual), pulmonary, vaginal, urethral, and rectal administration. Administration can also be via nasal spray, surgical implant, internal surgical paint, infusion pump, or via catheter, stent, balloon or other delivery device. The most useful and/or beneficial mode of administration can vary, especially depending upon the condition of the recipient and the disorder being treated. In preferred embodiments, the compositions of the present invention are provided in an oral dosage form, as more fully described below.

The pharmaceutical compositions may be conveniently made available in a unit dosage form, whereby such compositions may be prepared by any of the methods generally known in the pharmaceutical arts. Generally speaking, such methods of preparation comprise combining (by various methods) the active compounds of the invention with a suitable carrier or other adjuvant, which may consist of one or more ingredients. The combination of the active ingredients with the one or more adjuvants is then physically treated to present the composition in a suitable form for delivery (e.g., shaping into a tablet or forming an aqueous suspension).

Pharmaceutical compositions according to the present invention suitable for oral dosage may take various forms, such as tablets, capsules, caplets, and wafers (including rapidly dissolving or effervescing), each containing a predetermined amount of the active agent. The compositions may also be in the form of a powder or granules, a solution or suspension in an aqueous or non-aqueous liquid, and as a liquid emulsion (oil-in-water and water-in-oil). The active agents may also be delivered as a bolus, electuary, or paste. It is generally understood that methods of preparations of the above dosage forms are generally known in the art, and any such method would be suitable for the preparation of the respective dosage forms for use in delivery of the compositions according to the present invention.

In one embodiment, compound may be administered orally in combination with a pharmaceutically acceptable vehicle such as an inert diluent or an edible carrier. Oral compositions may be enclosed in hard or soft shell gelatin capsules, may be compressed into tablets or may be incorporated directly with the food of the patient's diet. The percentage of the composition and preparations may be varied; however, the amount of substance in such therapeutically useful compositions is preferably such that an effective dosage level will be obtained.

Hard capsules containing the compound may be made using a physiologically degradable composition, such as gelatin. Such hard capsules comprise the compound, and may further comprise additional ingredients including, for example, an inert solid diluent such as calcium carbonate, calcium phosphate, or kaolin. Soft gelatin capsules containing the compound may be made using a physiologically degradable composition, such as gelatin. Such soft capsules comprise the compound, which may be mixed with water or an oil medium such as peanut oil, liquid paraffin, or olive oil.

Sublingual tablets are designed to dissolve very rapidly. Examples of such compositions include ergotamine tartrate, isosorbide dinitrate, and isoproterenol HCL. The compositions of these tablets contain, in addition to the drug, various soluble excipients, such as lactose, powdered sucrose, dextrose, and mannitol. The solid dosage forms of the present invention may optionally be coated, and examples of suitable coating materials include, but are not limited to, cellulose polymers (such as cellulose acetate phthalate, hydroxypropyl cellulose, hydroxypropyl methylcellulose, hydroxypropyl methylcellulose phthalate, and hydroxypropyl methylcellulose acetate succinate), polyvinyl acetate phthalate, acrylic acid polymers and copolymers, and methacrylic resins (such as those commercially available under the trade name EUDRAGIT®), zein, shellac, and polysaccharides.

Powdered and granular compositions of a pharmaceutical preparation of the invention may be prepared using known methods. Such compositions may be administered directly to a patient or used in the preparation of further dosage forms, such as to form tablets, fill capsules, or prepare an aqueous or oily suspension or solution by addition of an aqueous or oily vehicle thereto. Each of these compositions may further comprise one or more additives, such as dispersing or wetting agents, suspending agents, and preservatives. Additional excipients (e.g., fillers, sweeteners, flavoring, or coloring agents) may also be included in these compositions.

Liquid compositions of the pharmaceutical composition of the invention which are suitable for oral administration may be prepared, packaged, and sold either in liquid form or in the form of a dry product intended for reconstitution with water or another suitable vehicle prior to use.

A tablet containing one or more compounds according to the present invention may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agents.

Adjuvants or accessory ingredients, in addition to those discussed above, for use in the compositions of the present invention can include any pharmaceutical ingredient commonly deemed acceptable in the art, such as binders, fillers, lubricants, disintegrants, diluents, surfactants, stabilizers, preservatives, flavoring and coloring agents, and the like. Binders are generally used to facilitate cohesiveness of the tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluloses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the composition according to the present invention may be anionic, cationic, amphoteric, or nonionic surface active agents. Stabilizers may be included in the compositions to inhibit or lessen reactions leading to decomposition of the active agents, such as oxidative reactions.

Solid dosage forms may be formulated so as to provide a delayed release of the active agents, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the active agents over a prolonged period of time), and may or may not also be delayed release. Sustained release compositions are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

In certain embodiments, the compounds and compositions disclosed herein can be delivered via a medical device. Such delivery can generally be via any insertable or implantable medical device, including, but not limited to stents, catheters, balloon catheters, shunts, or coils. In one embodiment, the present invention provides medical devices, such as stents, the surface of which is coated with a compound or composition as described herein. The medical device of this invention can be used, for example, in any application for treating, preventing, or otherwise affecting the course of a disease or condition, such as those disclosed herein.

In another embodiment of the invention, the pharmaceutical compositions of the invention can be administered intermittently. Administration of the therapeutically effective dose may be achieved in a continuous manner, as for example with a sustained-release composition, or it may be achieved according to a desired daily dosage regimen, as for example with one, two, three, or more administrations per day. By “time period of discontinuance” is intended a discontinuing of the continuous sustained-released or daily administration of the composition. The time period of discontinuance may be longer or shorter than the period of continuous sustained-release or daily administration. During the time period of discontinuance, the level of the components of the composition in the relevant tissue is substantially below the maximum level obtained during the treatment. The preferred length of the discontinuance period depends on the concentration of the effective dose and the form of composition used. The discontinuance period can be at least 2 days, at least 4 days or at least 1 week. In other embodiments, the period of discontinuance is at least 1 month, 2 months, 3 months, 4 months or greater. When a sustained-release composition is used, the discontinuance period must be extended to account for the greater residence time of the composition in the body. Alternatively, the frequency of administration of the effective dose of the sustained-release composition can be decreased accordingly. An intermittent schedule of administration of a composition of the invention can continue until the desired therapeutic effect, and ultimately treatment of the disease or disorder, is achieved.

The inventive pharmaceutical compositions can comprise a single polymorph as described herein, can comprise two or more polymorphs as described herein, or can comprise one or more polymorphs as described herein with one or more further pharmaceutically active compounds (i.e., co-administration). Accordingly, it is recognized that the pharmaceutically active compounds in the compositions of the invention can be administered in a fixed combination (i.e., a single pharmaceutical composition that contains both active materials). Alternatively, the pharmaceutically active compounds may be administered simultaneously (i.e., separate compositions administered at the same time). In another embodiment, the pharmaceutically active compounds are administered sequentially (i.e., administration of one or more pharmaceutically active compounds followed by separate administration or one or more pharmaceutically active compounds). One of skill in the art will recognized that the most preferred method of administration will allow the desired therapeutic effect.

Delivery of a therapeutically effective amount of a composition according to the invention may be obtained via administration of a therapeutically effective dose of the composition. Accordingly, in one embodiment, a therapeutically effective amount is an amount effective to treat abnormal cell proliferation. In another embodiment, a therapeutically effective amount is an amount effective to treat inflammation. In yet another embodiment, a therapeutically effective amount is an amount effective to treat arthritis. In still another embodiment, a therapeutically effective amount is an amount effective to treat asthma.

The active compound is included in the pharmaceutical composition in an amount sufficient to deliver to a patient a therapeutic amount of a compound of the invention in vivo in the absence of serious toxic effects. The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time

A therapeutically effective amount according to the invention can be determined based on the bodyweight of the recipient. For example, in one embodiment, a therapeutically effective amount of one or more compounds of the invention is in the range of about 0.1 μg/kg of body weight to about 5 mg/kg of body weight per day. Alternatively, a therapeutically effective amount can be described in terms of a fixed dose. Therefore, in another embodiment, a therapeutically effective amount of one or more compounds of the invention is in the range of about 0.01 mg to about 500 mg per day. Of course, it is understood that such an amount could be divided into a number of smaller dosages administered throughout the day.

It is contemplated that the compositions of the invention comprising one or more compounds described herein will be administered in therapeutically effective amounts to a mammal, preferably a human. An effective dose of a compound or composition for treatment of any of the conditions or diseases described herein can be readily determined by the use of conventional techniques and by observing results obtained under analogous circumstances. The effective amount of the compositions would be expected to vary according to the weight, sex, age, and medical history of the subject. Of course, other factors could also influence the effective amount of the composition to be delivered, including, but not limited to, the specific disease involved, the degree of involvement or the severity of the disease, the response of the individual patient, the particular compound administered, the mode of administration, the bioavailability characteristics of the preparation administered, the dose regimen selected, and the use of concomitant medication. The compound is preferentially administered for a sufficient time period to alleviate the undesired symptoms and the clinical signs associated with the condition being treated. Methods to determine efficacy and dosage are known to those skilled in the art. See, for example, Isselbacher et al. (1996) Harrison's Principles of Internal Medicine 13 ed., 1814-1882, herein incorporated by reference.

### IV. Active Agent Combinations

For use in treating various diseases or conditions, the pharmaceutical compositions of the invention can include the polymorphs described above in various combinations. For example, in one embodiment, a pharmaceutical composition according to the invention can comprise a single polymorph of the compound of Formula (1). In another embodiment, a pharmaceutical composition according to the invention can comprise two or more polymorphs of the compound of Formula (1). In still further embodiments, a pharmaceutical composition according to the invention can comprise one or more polymorphs of the compound of Formula (1) with one or more further compounds recognized as having therapeutic properties. For example, the pharmaceutical compositions described herein can be administered with one or more toxicity-reducing compounds (e.g., folic acid or leucovorin). In further embodiments, the inventive pharmaceutical compositions can be administered with one or more compounds known to be an anti-inflammatory, anti-arthritic, antibiotic, antifungal, or antiviral agent. Such further compounds can be provided as a component of the pharmaceutical composition or can be provided in alternation with the compositions of the invention. In other words, the pharmaceutical compositions of the invention can be administered with the additional active agent(s) in the same composition with the polymorph of the compound of Formula (1), or the additional active agent(s) can be administered in a separate delivery form from the pharmaceutical compositions of the invention. In particular embodiments, the pharmaceutical compositions of the invention can be provided in combination with one or more compounds selected from the groups described below.

In the following description, certain compounds useful as further active agents in the pharmaceutical compositions of the invention with the polymorphs disclosed above may be described in reference to specific diseases or conditions commonly treated using the noted compounds. The disclosure of such diseases or conditions is not intended to limit the scope of the invention and particularly does not limit the diseases or conditions that may be treated using the pharmaceutical compositions disclosed herein. Rather such exemplary diseases or conditions are provided only to illustrate the types of diseases and conditions typically treated using the additional compounds.

As additional active agents, the pharmaceutical compositions of the present invention can, in certain embodiments, be administered with antiproliferative agents. Proliferative disorders are currently treated by a variety of classes of compounds including alkylating agents, antimetabolites, natural products, enzymes, biological response modifiers, miscellaneous agents, radiopharmaceuticals (for example, Y-90 tagged to hormones or antibodies), hormones and antagonists. Any of the antiproliferative agents listed below or any other such therapeutic agents and principles as described in, for example, DeVita, V. T., Jr., Hellmann, S., Rosenberg, S. A.; Cancer: Principles & Practice of Oncology, 5th ed., Lippincott-Raven Publishers (1997), can be used with the pharmaceutical compositions of the present invention

Representative, nonlimiting examples of anti-angiogenesis agents suitable for use with the pharmaceutical compositions of the present invention include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs (I-azetidine-2-carboxylic acid (LACA), cis-hydroxyproline), d,1-3,4-dehydroproline, thiaproline, alpha,alpha-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chimp-3, chymostatin, beta-cyclodextrin tetradecasulfate, eponemycin, fumagillin, gold sodium thiomalate, d-penicillamine (CDPT), beta-1-anticollagenase-serum, alpha-2-antiplasmin, bisantrene, lobenzarit disodium, n-(2-carboxyphenyl-4-chloroanthronilic acid disodium or “CCA”, thalidomide, angostatic steroid, cargboxynaminolmidazole, and metalloproteinase inhibitors such as BB94. Other anti-angiogenesis agents include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

Representative, nonlimiting examples of alkylating agents suitable for use with the pharmaceutical compositions of the present invention include, but are not limited to, Nitrogen Mustards, such as Mechlorethamine (Hodgkin's disease, non-Hodgkin's lymphomas), Cyclophosphamide, Ifosfamide (acute and chronic lymphocytic leukemias, Hodgkin's disease, non-Hodgkin's lymphomas, multiple myeloma, neuroblastoma, breast, ovary, lung, Wilms' tumor, cervix, testis, soft-tissue sarcomas), Melphalan (L-sarcolysin) (multiple myeloma, breast, ovary), Chlorambucil (chronic lymphocytic leukemia, primary macroglobulinemia, Hodgkin's disease, non-Hodgkin's lymphomas), Ethylenimines and Methylmelamines, such as, Hexamethylmelamine (ovary), Thiotepa (bladder, breast, ovary), Alkyl Sulfonates, such as, Busulfan (chronic granulocytic leukemia), Nitrosoureas, such as, Carmustine (BCNU) (Hodgkin's disease, non-Hodgkin's lymphomas, primary brain tumors, multiple myeloma, malignant melanoma), Lomustine (CCNU) (Hodgkin's disease, non-Hodgkin's lymphomas, primary brain tumors, small-cell lung), Semustine (methyl-CCNU) (primary brain tumors, stomach, colon), Streptozocin (STR) (malignant pancreatic insulinoma, malignant carcinoin) and Triazenes, such as, Dacarbazine (DTIC-dimethyltriazenoimidazole-carboxamide) (malignant melanoma, Hodgkin's disease, soft-tissue sarcomas).

Representative, nonlimiting examples of anti-metabolite agents suitable for use with the pharmaceutical compositions of the present invention include, but are not limited to, Folic Acid Analogs, such as, Methotrexate (amethopterin) (acute lymphocytic leukemia, choriocarcinoma, mycosis fungoides, breast, head and neck, lung, osteogenic sarcoma), Pyrimidine Analogs, such as Fluorouracil (5-fluorouracil-5-FU) Floxuridine (fluorodeoxyuridine-FUdR) (breast, colon, stomach, pancreas, ovary, head and neck, urinary bladder, premalignant skin lesions) (topical), Cytarabine (cytosine arabinoside) (acute granulocytic and acute lymphocytic leukemias), Purine Analogs and Related Inhibitors, such as, Mercaptopurine (6-mercaptopurine-6-MP) (acute lymphocytic, acute granulocytic and chronic granulocytic leukemia), Thioguanine (6-thioguanine-TG) (acute granulocytic, acute lymphocytic and chronic granulocytic leukemia), Pentostatin (2′-deoxycyoformycin) (hairy cell leukemia, mycosis fungoides, chronic lymphocytic leukemia), Vinca Alkaloids, such as, Vinblastine (VLB) (Hodgkin's disease, non-Hodgkin's lymphomas, breast, testis), Vincristine (acute lymphocytic leukemia, neuroblastoma, Wilms' tumor, rhabdomyosarcoma, Hodgkin's disease, non-Hodgkin's lymphomas, small-cell lung), Epipodophylotoxins, such as Etoposide (testis, small-cell lung and other lung, breast, Hodgkin's disease, non-Hodgkin's lymphomas, acute granulocytic leukemia, Kaposi's sarcoma), and Teniposide (testis, small-cell lung and other lung, breast, Hodgkin's disease, non-Hodgkin's lymphomas, acute granulocytic leukemia, Kaposi's sarcoma).

Representative, nonlimiting examples of cytotoxic agents suitable for use with the pharmaceutical compositions of the present invention include, but are not limited to: doxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci.

Representative, non-limiting examples of natural products suitable for use with the pharmaceutical compositions of the present invention include, but are not limited to: Antibiotics, such as, Dactinomycin (actinonmycin D) (choriocarcinoma, Wilms' tumor rhabdomyosarcoma, testis, Kaposi's sarcoma), Daunorubicin (daunomycin-rubidomycin) (acute granulocytic and acute lymphocytic leukemias), Doxorubicin (soft tissue, osteogenic, and other sarcomas, Hodgkin's disease, non-Hodgkin's lymphomas, acute leukemias, breast, genitourinary thyroid, lung, stomach, neuroblastoma), Bleomycin (testis, head and neck, skin and esophagus lung, and genitourinary tract, Hodgkin's disease, non-Hodgkin's lymphomas), Plicamycin (mithramycin) (testis, malignant hypercalcemia), Mitomycin (mitomycin C) (stomach, cervix, colon, breast, pancreas, bladder, head and neck), Enzymes, such as, L-Asparaginase (acute lymphocytic leukemia), and Biological Response Modifiers, such as, Interferon-alpha (hairy cell leukemia, Kaposi's sarcoma, melanoma, carcinoid, renal cell, ovary, bladder, non Hodgkin's lymphomas, mycosis fungoides, multiple myeloma, chronic granulocytic leukemia).

Additional agents that can be used with the pharmaceutical compositions disclosed herein include, but are not limited to: Platinum Coordination Complexes, such as, Cisplatin (cis-DDP) Carboplatin (testis, ovary, bladder, head and neck, lung, thyroid, cervix, endometrium, neuroblastoma, osteogenic sarcoma); Anthracenedione, such as

Mixtozantrone (acute granulocytic leukemia, breast); Substituted Urea, such as, Hydroxyurea (chronic granulocytic leukemia, polycythemia vera, essential thrombocytosis, malignant melanoma); Methylhydrazine Derivatives, such as, Procarbazine (N-methylhydrazine, MIH) (Hodgkin's disease); Adrenocortical Suppressants, such as, Mitotane (o,p′-DDD) (adrenal cortex), Aminoglutethimide (breast); Adrenorticosteriods, such as, Prednisone (acute and chronic lymphocytic leukemias, non-Hodgkin's lymphomas, Hodgkin's disease, breast); Progestins, such as, Hydroxprogesterone caproate, Medroxyprogesterone acetate, Megestrol acetate (endometrium, breast); and Steroids, such as betamethasone sodium phosphate and betamethasone acetate.

Representative, nonlimiting examples of hormones and antagonists suitable for use with the pharmaceutical compositions of the present invention include, but are not limited to, Estrogens: Diethylstibestrol Ethinyl estradiol (breast, prostate); Antiestrogen: Tamoxifen (breast); Androgens: Testosterone propionate Fluxomyesterone (breast); Antiandrogen: Flutamide (prostate); Gonadotropin-Releasing Hormone Analog: and Leuprolide (prostate). Other hormones include medroxyprogesterone acetate, estradiol, megestrol acetate, octreotide acetate, diethylstilbestrol diphosphate, testolactone, and goserelin acetate.

The pharmaceutical compositions of the present invention can be used with therapeutic agents used to treat arthritis. Examples of such agents include, but are not limited to, the following:

Nonsteroidal anti-inflammatory drugs (NSAIDs), such as cylcooxygenase-2 (COX-2) inhibitors, aspirin (acetylsalicylic acid), ibuprofen, ketoprofen, naproxen, and acetaminophen;

Analgesics, such as acetaminophen, opioid analgesics, and transdermal fentanyl;

Biological response modifiers, such as etanercept, infliximab, adalimumab, anakinra, abatacept, tiruximab, certolizumab pegol, and tocilizumab;

Corticosteroids or steroids, such as glucocorticoids (GC), fluticasone, budesonide, prednisolone, hydrocortisone, adrenaline, Aldosterone, Cortisone Acetate, Desoxymethasone, Dexamethasone, Fluocortolone, Hydrocortisone, Meprednisone, Methylprednisolone, Prednisolone, Prednisone, Prednylidene, Procinonide, Rimexolone, and Suprarenal Cortex;

Disease-modifying antirheumatic drugs (DMARDs), such as hydroxychloroquine, cyclosphosphamide, chlorambucil, the gold compound auranofin, sulfasalazine, minocycline, cyclosporine, toll-like receptor agonists and antagonists, kinase inhibitors (e.g., p38 MAPK) immunosuppressants and tumor necrosis factor (TNF) blockers (e.g., etanercept, infliximab, and adalimumab);

Fibromyalgia medications, such as amitriptyline, fluoxetine, cylobenzaprine, tramadol, gabapentin, pregabalin, and dual-reuptake inhibitors;

Osteoporosis medications, such as estrogens, parathyroid hormones, bisphosphonates, selective receptor molecules, and bone formation agents;

Gout medications, such as allopurinol, probenecid, losartan, and fenofibrate;

Psoriasis medications, such as acitretin; and

Topical treatments, such as topical NSAIDs and capsaicin.

The pharmaceutical compositions of the present invention also can be used with therapeutic agents used to treat asthma. Examples of such agents include, but are not limited to, the following:

Anti-allergics, such as cromolyn sodium and ketotifen fumarate;

Anti-inflammatories, such as NSAIDs and steroidal anti-inflammatories (e.g., beclomethasone dipropionate, budesonide, dexamethasone sodium phosphate, flunisolide, fluticasone propionate, and triamcinolone acetonide);

Anticholinergics, such as ipratropium bromide, belladonna alkaloids, atropine, and oxitropium bromide;

Antihistamines, such as chlorpheniramine, brompheniramine, diphenhydramine, clemastine, dimenhydrinate, cetirizine, hydroxyzine, meclizine, fexofenadine, loratadine, and enadine;

β₂-adrenergic agonists (beta agonists), such as albutamol, terbutaline, epinephrine, metaproterenol, ipratropium bromide, ephedra (source of alkaloids), ephedrine, and psuedoephedrine;

Leukotriene Receptor Antagonists, such as zafirlukast and zileuton montelukast;

Xanthines (bronchodilators), such as theophylline, dyphylline, and oxtriphylline; Miscellaneous anti-asthma agents, such as xanthines, methylxanthines, oxitriphylline, aminophylline, phosphodiesterase inhibitors such as zardaverine, calcium antagonists such as nifedipine, and potassium activators such as cromakalim; and

Prophylactic agent(s), such as sodium cromoglycate, cromolyn sodium, nedocromil, and ketotifen.

Further, non-limiting examples of active agents that can be used with the pharmaceutical compositions of the present invention include anti-psoriasis agents, anti-Inflammatory Bowel Disease (anti-IBD) agents, anti-chronic obstructive pulmonary disease (anti-COPD) agents, anti-multiple sclerosis agents.

In specific embodiments, the polymorphs of the present invention can be combined with methotrexate. Specifically, the compounds may be administered in a combination, such as being provided together in the same dosage form or as being provided in separate dosage forms intended to be used together in a combination therapy (e.g., the polymorph being administered on a first schedule and the methotrexate being administered on a second schedule). The two schedules particularly may overlap.

### V. Articles of Manufacture

The present invention also includes an article of manufacture providing a pharmaceutical composition comprising one or more polymorphs of the compound of Formula (1), optionally in combination with one or more further active agents. The article of manufacture can include a vial or other container that contains a composition suitable for use according to the present invention together with any carrier, either dried or in liquid form. In particular, the article of manufacture can comprise a kit including a container with a composition according to the invention. In such a kit, the composition can be delivered in a variety of combinations. For example, the composition can comprise a single dosage comprising all of the active ingredients. Alternately, where more than one active ingredient is provided, the composition can comprise multiple dosages, each comprising one or more active ingredients, the dosages being intended for administration in combination, in succession, or in other close proximity of time. For example, the dosages could be solid forms (e.g., tablets, caplets, capsules, or the like) or liquid forms (e.g., vials), each comprising a single active ingredient, but being provided in blister packs, bags, or the like, for administration in combination. In specific embodiments, the dosage form with the first active ingredient (e.g., a polymorph according to the invention) could be provided for daily administration, and the dosage form with the second active ingredient (e.g., methotrexate or another compound) could be provided for weekly administration.

The article of manufacture further can include instructions in the form of a label on the container and/or in the form of an insert included in a box in which the container is packaged, for the carrying out the method of the invention. The instructions can also be printed on the box in which the vial is packaged. The instructions contain information such as sufficient dosage and administration information so as to allow the subject or a worker in the field to administer the pharmaceutical composition. It is anticipated that a worker in the field encompasses any doctor, nurse, technician, spouse, or other caregiver that might administer the composition. The pharmaceutical composition can also be self-administered by the subject.

### VI. Methods of Treatment

The disclosed polymorphs of the compound of Formula (1) can be useful in the treatment of various conditions wherein disruption of folic acid metabolism is beneficial for treating a symptom of the condition or the condition generally. Accordingly, in further embodiments, the present invention is directed to methods of treating various diseases or conditions. In particular embodiments, the invention provides methods of treating diseases or conditions known or found to be treatable by disruption of folic acid metabolism. In specific embodiments, the invention provides methods of treating conditions, such as abnormal cell proliferation, inflammation (including inflammatory bowel disease), arthritis (particularly rheumatoid arthritis), psoriasis, and asthma.

A. Abnormal Cellular Proliferation

Abnormal cell proliferation has been shown to be the root of many diseases and conditions, including cancer and non-cancer disorders which present a serious health threat. Generally, the growth of the abnormal cells, such as in a tumor, exceeds and is uncoordinated with that of normal cells. Furthermore, the abnormal growth of tumor cells generally persists in an abnormal (i.e., excessive) manner after the cessation of stimuli that originally caused the abnormality in the growth of the cells. A benign tumor is characterized by cells that retain their differentiated features and do not divide in a completely uncontrolled manner. A benign tumor is usually localized and nonmetastatic. A malignant tumor (i.e. , cancer) is characterized by cells that are undifferentiated, do not respond to the body's growth control signals, and multiply in an uncontrolled manner.

Malignant tumors are invasive and capable of metastasis.

Treatment of diseases or conditions of abnormal cellular proliferation comprises methods of killing, inhibiting, or slowing the growth or increase in size of a body or population of abnormally proliferative cells (including tumors or cancerous growths), reducing the number of cells in the population of abnormally proliferative cells, or preventing the spread of abnormally proliferative cells to other anatomic sites, as well as reducing the size of a growth of abnormally proliferative cells. The term “treatment” does not necessarily mean to imply a cure or a complete abolition of the disorder of abnormal cell proliferation. Prevention of abnormal cellular proliferation comprises methods which slow, delay, control, or decrease the likelihood of the incidence or onset of disorders of abnormal cell proliferation, in comparison to that which would occur in the absence of treatment.

Abnormal cellular proliferation, notably hyperproliferation, can occur as a result of a wide variety of factors, including genetic mutation, infection, exposure to toxins, autoimmune disorders, and benign or malignant tumor induction. Hyperproliferative cell disorders include, but are not limited to, skin disorders, blood vessel disorders, cardiovascular disorders, fibrotic disorders, mesangial disorders, autoimmune disorders, graft-versus-host rejection, tumors, and cancers.

Representative, non-limiting types of non-neoplastic abnormal cellular proliferation disorders that can be treated using the present invention include: skin disorders such as psoriasis, eczerma, keratosis, basal cell carcinoma, and squamous cell carcinoma; disorders of the cardiovascular system such as hypertension and vasculo-occlusive diseases (e.g., atherosclerosis, thrombosis and restenosis); blood vessel proliferative disorders such as vasculogenic (formation) and angiogenic (spreading) disorders which result in abnormal proliferation of blood vessels, such as antiogenesis; and disorders associated with the endocrine system such as insulin resistant states including obesity and diabetes mellitus (types 1 & 2).

The compositions and methods of the present invention are also useful for treating inflammatory diseases associated with non-neoplastic abnormal cell proliferation. These include, but are not limited to, inflammatory bowel disease (IBD), rheumatoid arthritis (RA), multiple sclerosis (MS), proliferative glomerulonephritis, lupus erythematosus, scleroderma, temporal arteritis, thromboangiitis obliterans, mucocutaneous lymph node syndrome, asthma, host versus graft, thyroiditis, Grave's disease, antigen-induced airway hyperactivity, pulmonary eosinophilia, Guillain-Barre syndrome, allergic rhinitis, myasthenia gravis, human T-lymphotrophic virus type 1-associated myelopathy, herpes simplex encephalitis, inflammatory myopathies, atherosclerosis, and Goodpasture's syndrome.

In a particular embodiment, the polymorphs of the present invention are useful in the treatment of psoriasis. Psoriasis is an immune-mediated skin disorder characterized by chronic T-cell stimulation by antigen-presenting cells (APC) occurs in the skin. The various types of psoriasis include, for example, plaque psoriasis (i.e., vulgaris psoriasis), pustular psoriasis, guttate psoriasis, inverse psoriasis, erythrodermic psoriasis, psoriatic arthritis, scalp psoriasis and nail psoriasis. Common systemic treatments for psoriasis include methotrexate, cyclosporin and oral retinoids, but their use is limited by toxicity. Up to 40% of patients with psoriasis also develop psoriatic arthritis (Kormeili T et al. Br J Dermatol. (2004) 151(1):3-15.

In further embodiments, the polymorphs of the present invention are useful in the treatment of blood vessel proliferative disorders, including vasculogenic (formation) and angiogenic (spreading) disorders which result in abnormal proliferation of blood vessels. Other blood vessel proliferative disorders include arthritis and ocular diseases such as diabetic retinopathy. Abnormal neovascularization is also associated with solid tumors.

In a particular embodiment, the compositions of the present invention are useful in the treatment of diseases associated with uncontrolled angiogenesis. Representative, non-limiting diseases of abnormal angiogenesis include rheumatoid arthritis, ischemic-reperfusion related brain edema and injury, cortical ischemia, ovarian hyperplasia and hypervascularity, (polycystic ovary syndrome), endometriosis, psoriasis, diabetic retinopathy, and other ocular angiogenic diseases such as retinopathy of prematurity (retrolental fibroplastic), macular degeneration, corneal graft rejection, neuroscular glaucoma, and Oster Webber syndrome. Cancers associated with abnormal blood cell proliferation include hemangioendotheliomas, hemangiomas, and Kaposi's sarcoma.

In further embodiments, the polymorphs of the present invention are useful in the treatment of disorders of the cardiovascular system involving abnormal cell proliferation. Such disorders include, for example, hypertension, vasculo-occlusive diseases (e.g., atherosclerosis, thrombosis, and restenosis after angioplasty), acute coronary syndromes (such as unstable angina, myocardial infarction, ischemic and non-ischemic cardiomyopathies, post-MI cardiomyopathy, and myocardial fibrosis), and substance-induced cardiomyopathy.

Vascular injury can also result in endothelial and vascular smooth muscle cell proliferation. The injury can be caused by traumatic events or interventions (e.g., angioplasty, vascular graft, anastomosis, organ transplant) (Clowes A et al. A. J. Vasc. Surg (1991) 13:885). Restenosis (e.g., coronary, carotid, and cerebral lesions) is the main complication of successful balloon angioplasty of the coronary arteries. It is believed to be caused by the release of growth factors as a result of mechanical injury to the endothelial cells lining the coronary arteries.

Other atherosclerotic conditions which can be treated or prevented by means of the present invention include diseases of the arterial walls that involve proliferation of endothelial and/or vascular smooth muscle cells, including complications of diabetes, diabetic glomerulosclerosis, and diabetic retinopathy.

In further embodiments, the polymorphs of the present invention are useful in the treatment of abnormal cell proliferation disorders associated the endocrine system. Such disorders include, for example, insulin resistant states including obesity, diabetes mellitus (types 1 & 2), diabetic retinopathy, macular degeneration associated with diabetes, gestational diabetes, impaired glucose tolerance, polycystic ovarian syndrome, osteoporosis, osteopenia, and accelerated aging of tissues and organs including Wemer's syndrome.

In further embodiments, the polymorphs of the present invention are useful in the treatment of abnormal cell proliferation disorders of the urogenital system. These include, for example, edometriosis, benign prostatic hyperplasia, eiomyoma, polycystic kidney disease, and diabetic nephropathy.

In further embodiments, the polymorphs of the present invention are useful in the treatment of fibrotic disorders. Medical conditions involving fibrosis include undesirable tissue adhesion resulting from surgery or injury. Non-limiting examples of fibrotic disorders include hepatic cirrhosis and mesangial proliferative cell disorders.

In still further embodiments, abnormal cell proliferation disorders of the tissues and joints can be treated according to the present invention. Such disorders include, for example, Raynaud's phenomenon/disease, Sjogren's Syndrome systemic sclerosis, systemic lupus erythematosus, vasculitides, ankylosing spondylitis, osteoarthritis, reactive arthritis, psoriatic arthritis, and fibromyalgia.

In certain embodiments, abnormal cell proliferation disorders of the pulmonary system can also be treated according to the present invention. These disorders include, for example, asthma, chronic obstructive pulmonary disease (COPD), reactive airway disease, pulmonary fibrosis, and pulmonary hypertension.

Further disorders including an abnormal cellular proliferative component that can be treated according to the invention include Behcet's syndrome, fibrocystic breast disease, fibroadenoma, chronic fatigue syndrome, acute respiratory distress syndrome (ARDS), ischemic heart disease, post-dialysis syndrome, leukemia, acquired immune deficiency syndrome, vasculitis, lipid histiocytosis, septic shock, and familial intestinal polyposes such as Gardner syndrome. Also included in the scope of disorders that may be treated by the compositions and methods of the present invention are virus-induced hyperproliferative diseases including, for example, human papilloma virus-induced disease (e.g., lesions caused by human papilloma virus infection), Epstein-Barr virus-induced disease, scar formation, genital warts, cutaneous warts, and the like.

The polymorphs of the present invention are further useful in the treatment of conditions and diseases of abnormal cell proliferation including various types of cancers such as primary tumors and tumor metastasis. Specific, non-limiting types of benign tumors that can be treated according to the present invention include hemangiomas, hepatocellular adenoma, cavernous hemangiomas, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas, and pyogenic granulomas.

Representative, non-limiting cancers treatable according to the invention include breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, reticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, epidermoid carcinomas, and other carcinomas and sarcomas.

The polymorphs of the present invention are also useful in preventing or treating proliferative responses associated with organ transplantation which contribute to rejections or other complications. For example, proliferative responses may occur during transplantation of the heart, lung, liver, kidney, and other body organs or organ systems.

B. Inflammation

The polymorphs of the present invention are also useful in the treatment of diseases characterized by inflammation. Diseases and conditions which have significant inflammatory components are ubiquitous and include, for example, skin disorders, bowel disorders, certain degenerative neurological disorders, arthritis, autoimmune diseases and a variety of other illnesses. Some of these diseases have both an inflammatory and proliferative component, as described above. In particular embodiments the compounds are used to treat inflammatory bowel diseases (IBD), Crohn's disease (CD), ulcerative colitis (UC), chronic obstructive pulmonary disease (COPD), sarcoidosis, or psoriasis. The disclosed polymorphs are also useful in the treatment of other inflammatory diseases, for example, allergic disorders, skin disorders, transplant rejection, poststreptococcal and autoimmune renal failure, septic shock, systemic inflammatory response syndrome (SIRS), adult respiratory distress syndrome (ARDS), envenomation, lupus erythematosus, Hashimoto's thyroiditis, autoimmune hemolytic anemias, insulin dependent diabetes mellitus, and rheumatic fever, pelvic inflammatory disease (PID), conjunctivitis, dermatitis, and bronchitis.

Inflammatory bowel diseases (IBD) includes several chronic inflammatory conditions, including Crohn's disease (CD) and ulcerative colitis (UC). Both CD and UC are considered “idiopathic” because their etiology is unknown. While Crohn's disease and ulcerative colitis share many symptoms (e.g., diarrhea, abdominal pain, fever, fatigue), ulcerative colitis is limited to the colon whereas Crohn's disease can involve any segment of the gastrointestinal tract. Both diseases may involve extraintestinal manifestations, including arthritis, diseases of the eye (e.g., episcleritis and iritis), skin diseases (e.g., erythema nodosum and pyoderma gangrenosum), urinary complications, gallstones, and anemia. Strokes, retinal thrombi, and pulmonary emboli are not uncommon, because many patients are in a hypercoagulable state.

In a particular embodiment, the polymorphs of the present invention are useful in the treatment of inflammatory bowel disease. In a preferred embodiment, the inflammatory bowel disease is Crohn's disease.

Chronic Obstructive Pulmonary Disease, or COPD, is characterized by a not fully reversible airflow limitation which is progressive and associated with an abnormal inflammatory reaction of the lungs. It is one of the most common respiratory conditions of adults, a major cause of chronic morbidity and mortality, and represents a substantial economic and social burden worldwide (Pauwels R A. Lancet. (2004) 364(9434):613-20). Other names for the disorder include, for example, Chronic Obstructive Airways Disease, (COAD); Chronic Obstructive Lung Disease, (COLD), Chronic Airflow Limitation, (CAL or CAFL) and Chronic Airflow Obstruction (COA).

COPD is characterized by chronic inflammation throughout the airways, parenchyma, and pulmonary vasculature. The inflammation involves a multitude of cells, mediators, and inflammatory effects. Mediators include, for example, mediators include proteases, oxidants and toxic peptides. Over time, inflammation damages the lungs and leads to the pathologic changes characteristic of COPD. Manifestations of disease includes both chronic bronchitis and emphysema. Chronic bronchitis is a long-standing inflammation of the airways that produces a lot of mucus, causing wheezing and infections. It is considered chronic if a subject has coughing and mucus on a regular basis for at least three months a year and for two years in a row. Emphysema is a disease that destroys the alveolae and/or bronchae, causing the air sacs to become enlarged, thus making breathing difficult. Most common in COPD patients is the centrilobular form of emphysema. In a particular embodiment, the compositions of the present invention are useful in the treatment of chronic obstructive pulmonary disease.

Sarcoidosis is yet another chronic inflammatory disease with associated abnormal cell proliferation. Sarcoidois is a multisystem granulomatous disorder wherein the granulomas are created by the angiogenic capillary sprouts providing a constant supply of inflammatory cells.

As noted above, inflammation also plays an important role in the pathogenesis of cardiovascular diseases, including restenosis, atherosclerotic complications resulting from plaque rupture, severe tissue ischemia, and heart failure. Inflammatory changes in the arterial wall, for example, are thought to play a major role in the development of restenosis and atherosclerosis (Ross R. N Engl J Med. (1999) 340: 115-126). Local inflammation occurs in the formation the plaques also contributes to the weakening of the fibrous cap of the advanced plaque, ultimately resulting in plaque rupture and acute coronary syndromes (Lind L. Atherosclerosis. (2003) 169(2):203-14).

Multiple sclerosis (MS) is a chronic, often debilitating autoimmune disease that affects the central nervous system. MS is characterized by inflammation which results when the body directs antibodies and white blood cells against proteins in the myelin sheath, fatty material which insulates the nerves in the brain and spinal cord. The result may be multiple areas of scarring (sclerosis), which slows or blocks muscle coordination, visual sensation and other nerve signals. In a particular embodiment, the polymorphs of the present invention are useful in the treatment of multiple sclerosis.

Inflammatory have been shown to be associated with the pathogenesis of neurological disorders, including Parkinson's disease and Alzheimer's disease (Mirza B. et al. Neuroscience (2000) 95(2):425-32; Gupta A. Int J Clin Pract. (2003) 57(1):36-9; Ghatan E. et al. Neurosci Biobehav Rev. (1999) 23(5):615-33).

The present invention is also useful in the treatment of, for example, allergic disorders, allergic rhinitis, skin disorders, transplant rejection, poststreptococcal and autoimmune renal failure, septic shock, systemic inflammatory response syndrome (SIRS), adult respiratory distress syndrome (ARDS), envenomation, lupus erythematosus, myasthenia gravis, Grave's disease, Hashimoto's thyroiditis, autoimmune hemolytic anemias, insulin dependent diabetes mellitus, glomerulonephritis, and rheumatic fever, pelvic inflammatory disease (PID), conjunctivitis, dermatitis, bronchitis, and rhinitis.

C. Asthma

In particular embodiments the polymorphs disclosed herein can be used in the treatment of asthma. In recent years, it has become clear that the primary underlying pathology of asthma is airway tissue inflammation (Lemanke (2002) Pediatrics 109(2):368-372; Nagayama et al. (1995) Pediatr Allergy Immunol. 6:204-208). Asthma is associated with a wide range of symptoms and signs, including wheezing, cough, chest tightness, shortness of breath and sputum production. Airway inflammation is a key feature of asthma pathogenesis and its clinical manifestations. Inflammatory cells, including mast cells, eosinophils, and lymphocytes, are present even in the airways of young patients with mild asthma.

Inflammation also plays a role in wheezing disorders, with or without asthma. Asthma is sometimes classified by the triggers that may cause an asthma episode (or asthma attack) or the things that make asthma worse in certain individuals, such as occupational asthma, exercise induced asthma, nocturnal asthma, or steroid resistant asthma. Thus, the polymorphs of the invention can also be used in the treatment of wheezing disorders, generally.

D. Arthritis and Osteoarthritis

More than 40 million Americans suffer from arthritis in its various forms, including over 100 kinds of rheumatic diseases (i.e., diseases affecting joints, muscle, and connective tissue, which makes up or supports various structures of the body, including tendons, cartilage, blood vessels, and internal organs). Representative types of arthritis include rheumatoid (such as soft-tissue rheumatism and non-articular rheumatism), fibromyalgia, fibrositis, muscular rheumatism, myofascil pain, humeral epicondylitis, frozen shoulder, Tietze's syndrome, fascitis, tendinitis, tenosynovitis, bursitis), juvenile chronic, spondyloarthropaties (ankylosing spondylitis), osteoarthritis, hyperuricemia and arthritis associated with acute gout, chronic gout, and systemic lupus erythematosus.

Hypertrophic arthritis or osteoarthritis is the most common form of arthritis and is characterized by the breakdown of the joint's cartilage. Osteoarthritis is common in people over 65, but may appear decades earlier. Breakdown of the cartilage causes bones to rub against each other, causing pain and loss of movement. In recent years, there has been increasing evidence that inflammation plays an important role in osteoarthritis. Nearly one-third of patients ready to undergo joint replacement surgery for osteoarthritis (OA) had severe inflammation in the synovial fluid that surrounds and protects the joints. In a particular embodiment, the polymorphs of the present invention are useful in the treatment of osteoarthritis.

The second most common form of arthritis is rheumatoid arthritis. It is an autoimmune disease that can affect the whole body, causing weakness, fatigue, loss of appetite, and muscle pain. Typically, the age of onset is much earlier than osteoarthritis, between ages 20 and 50. Inflammation begins in the synovial lining and can spread to the entire joint. Thus, the polymorphs of the invention are useful in the treatment of inflammatory diseases or conditions. In another embodiment, the polymorphs of the present invention are useful in the treatment of arthritis and specifically rheumatoid arthritis. In specific embodiments, inflammatory conditions, including arthritis (and particularly rheumatoid arthritis) may be treated according to the invention through administration of a polymorph described herein provided in some combination with another active agent. In particular embodiments, the polymorphs of the invention may be administered in combination with methotrexate. The methotrexate and the polymorphs may be administered in the same formulation, or they may be provided in separate formulations that are administered in combination (e.g., to be taken at or about the same time or to be taken at separate time that may or may not overlap—such as daily dosing for one formulation and weekly, bi-weekly, monthly, or one-time dosing for the other formulation).

### Experimental

The present invention will now be described with specific reference to various examples. The following examples are not intended to be limiting of the invention and are rather provided as exemplary embodiments.

### EXAMPLE 1

### Polymorph Screening—Methanol/Water Based Systems

Multiple different potential anti-solvents were tested using a mixture of methanol and water as the solvent for the compound of Formula (1). The compound of Formula (1) was dissolved using methanol and water in a 9:1 ratio. The solution was treated with activated carbon, filtered, and divided into 10 separate samples. Using a micro-scale analysis, different anti-solvents were added to the compound in the methanol/water solution. Each sample was evaluated for formation of crystalline product, and the results are provided below in Table 6.

TABLE 6

| Sample | Anti-Solvent | Result |
|---|---|---|
| 1 | THF | Crystalline Appearance |
| 2 | Acetone | Amorphous Appearance |
| 3 | Dioxane | Amorphous Appearance |
| 4 | Acetonitrile | Amorphous Appearance |
| 5 | Ethanol | Semi-crystalline |
| 6 | iso-Propanol | Semi-crystalline |
| 7 | Methyl Ethyl Ketone (MEK) | Crystalline Appearance |
| 8 | Methyl iso-Butyl Ketone (MiBK) | Crystalline Appearance |
| 9 | Methyl Acetate | Amorphous Appearance |
| 10 | Ethyl Acetate | Amorphous Appearance |

### EXAMPLE 2

### Polymorph Screening—Methanol Based Systems

Multiple different potential anti-solvents were tested using methanol as the solvent for the compound of Formula (1). The compound of Formula (1) was dissolved in methanol and the solution was treated with activated carbon, filtered, and divided into 9 separate samples. Using a micro-scale analysis, different anti-solvents were added to the compound in the methanol solution. Each sample was evaluated for formation of crystalline product, and the results are provided below in Table 7.

TABLE 7

| Sample | Anti-Solvent | Result |
|---|---|---|
| 1 | THF | Amorphous Appearance |
| 2 | Acetone | Amorphous Appearance |
| 3 | Dioxane | Amorphous Appearance |
| 4 | Acetonitrile | Amorphous Appearance |
| 5 | Ethanol | Amorphous Appearance |
| 6 | iso-Propanol | Amorphous Appearance |
| 7 | Methyl Ethyl Ketone (MEK) | Amorphous Appearance |
| 8 | Methyl iso-Butyl Ketone (MiBK) | Amorphous Appearance |
| 9 | Methanol | Crystalline Appearance |

### EXAMPLE 3

### Preparation of Polymorph Forms

The compound of Formula (1) (10 g) was mixed with activated carbon (1 g) and added to a mixture of methanol (45 mL) and water (5 mL). The solution was mixed for 30 minutes. The mixture was filtered through a SEITZ® K200 filter and then through a CELITE® pad (approximately 5 mm thickness). The resulting clear yellow solution was divided into 1.5 g portions The individual portions next were subjected to polymorphic screening with the addition of an anti-solvent (i.e., THF, MEK, or MiBK), as described below.

A 1.5 g amount of the above-obtained solution was combined with 1 g THF to form a slurry, to which was added an additional 1.5 g of the solution of the compound of Formula (1) and an additional 1 g of THF. The slurry was heated to about 40° C. and then allowed to cool to ambient temperature with stirring overnight. The resulting solid material was filtered and washed with THF/methanol (1:1, 1 mL) and THF (1 mL) to give 90 mg whitish solid polymorph II.

A 1.5 g amount of the above-obtained solution was combined with 0.5 g MEK to form a slurry, to which was added an additional 1.5 g of the solution of the compound of Formula (1) and an additional 0.6 g of MEK. The slurry was heated to about 40° C. and then allowed to cool to ambient temperature with stirring overnight. The resulting solid material was filtered and washed with MEK/methanol (1:1, 1 mL) and MEK (1 mL) to give 140 mg whitish solid polymorph Ia.

A 1.5 g amount of the above-obtained solution was combined with 0.5 g MIBK to form a slurry, to which was added an additional 1.5 g of the solution of the compound of

Formula (1) and an additional 0.6 g of MIBK. The slurry was heated to about 40° C. and then allowed to cool to ambient temperature with stirring overnight. The resulting solid material was filtered and washed with MIBK/methanol (1:1, 1 mL) and MIBK (1 mL) to give 240 mg whitish solid polymorph Ib.

### EXAMPLE 4

### Polymorph Stress Test

Samples of polymorph Form Ib and polymorph Form II were analyzed for purity by HPLC-UV according to method AM-5-ASP001 performed by gradient liquid chromatography with UV-detection at 237 nm. A Hypersil GOLD column from Thermo Scientific (250×4.6 mm, 5 μm) was used at a temperature of 30° C. A Dionex STH 585 column oven was used with a Dionex P580 gradient pump and a flow rate of 1.0 ml/min (gradient elution). A Dionex Degasys DG-1210 degasser and a Gynkotek Gina 50 autosampler injector were used with an injection volume of 5 μL. A Dionex PDA-100 photodiode array detector was used at a wavelength of 237 nm. Sample runtime was 46 minutes. The samples were tested on the day of synthesis (zero point) and at 1, 2, and 4 weeks storage at ambient conditions (i.e., room temperature and room humidity), at 25° C. (+/−2° C.) and 60% relative humidity (RH) (+/−5% RH), and at 40° C. (+/−2° C.) and 75 RH (+/−5% RH). Test results are provided in Table 8 (Form Ib) and Table 9 (Form II), wherein the values provided are the area %. Both polymorph forms were shown to be stable when stored at ambient conditions and when stored at 25° C. and 60% RH.

TABLE 8

| | Ambient Conditions | 25° C./60% RH | 40° C./75% RH |
|---|---|---|---|
| Zero point | 95.94 | — | — |
| 1 week | 95.87 | 95.96 | 95.87 |
| 2 weeks | 95.78 | 95.67 | 95.43 |
| 4 weeks | 95.91 | 95.85 | 95.34 |
| Average | 95.88 | 95.83 | 95.55 |

TABLE 9

| | Ambient Conditions | 25° C./60% RH | 40° C./75% RH |
|---|---|---|---|
| Zero point | 96.46 | — | — |
| 1 week | 96.42 | 96.53 | 96.43 |
| 2 weeks | 96.49 | 96.24 | 96.35 |
| 4 weeks | — | 96.24 | 95.52 |
| Average | 96.49 | 96.44 | 96.10 |

Samples of polymorph Form Ib and polymorph Form II also were analyzed for water content using Karl Fisher titration according to method AM-1-General/water content. A columetric Karl Fisher titrator (DL39, Mettler Toledo) was used with a Stromboli drying oven (Mettler Toledo), a generator electrode, and a standard analytical balance (at least 0.0 mg accuracy). Instrument conditions were set for an oven temperature of 150° C., a gas flow rate of 100.0 mL/min, and a stirrer speed of 50%. Control parameters were set for a max time of 600 s, a current of 2.0 μA, an end point of 100 mV, and a generation speed set at normal. The samples were tested on the day of synthesis (zero point) and at 1, 2, and 4 weeks storage at ambient conditions (i.e., room temperature and room humidity), at 25° C. (+/−2° C.) and 60% relative humidity (RH) (+/−5% RH), and at 40° C. (+/−2° C.) and 75% RH (+/−5% RH). Test results are provided in Table 10 (Form Ib) and Table 11 (Form II), wherein the values provided are the percent water content.

TABLE 10

| | Ambient Conditions | 25° C./60% RH | 40° C./75% RH |
|---|---|---|---|
| Zero point | 14.8 | — | — |
| 1 week | 16.2 | 16.0 | 19.6 |
| 2 weeks | 16.5 | 15.5 | 22.7 |
| 4 weeks | 18.4 | 16.4 | 26.8 |

TABLE 11

| | Ambient Conditions | 25° C./60% RH | 40° C./75% RH |
|---|---|---|---|
| Zero point | 15.6 | — | — |
| 1 week | 18.7 | 17.0 | 21.2 |
| 2 weeks | 21.1 | 16.3 | 24.4 |
| 4 weeks | 21.9 | 16.5 | 26.6 |

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A polymorph of the compound of Formula (1) 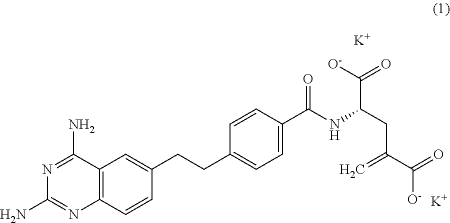
wherein the polymorph is a crystalline compound characterized by one or more of the following:
a) one or more of the approximate X-ray powder diffraction interplanar spacing peaks selected from the group consisting of 4.946, 7.118, 7.785, 8.238, 9.229, 9.822, 13.4000, 15.271, 15.658, 16.128, 16.459, 17.286, 18.088, 17.452, 18.889, 19.490, 19.837, 21.456, 22.658, 23.168, 23.811, 24.691, 28.436, and 29.609;
b) one or more of the approximate X-ray powder diffraction interplanar spacing peaks selected from the group consisting of 4.811, 8.316, 9.542, 10.047, 13.189, 14.946, 15.973, 17.219, 18.162, 21.814, 22.260, 23.087, 23.351, 24.518, 25.456, 26.846, 28.376, 29.648, 30.509, 31.226, and 32.328;
c) one or more of the approximate X-ray powder diffraction interplanar spacing peaks selected from the group consisting of 4.794, 7.020, 7.747, 8.104, 9.457, 11.483, 13.223, 15.010, 15.693, 16.943, 18.222, 19.552, 22.498, 23.003, and 29.490;
d) one or more of the approximate FTIR peaks selected from the group consisting of 3334, 3194, 1597, 1556, 1492, 1446, 1400, 1367, 1338, 1314, 1294, 1255, 1190, 1075, 1020, 992, 928, 835, 797, 748, and 733;
e) one or more of the approximate FTIR peaks selected from the group consisting of 332, 3207, 1555, 1499, 1443, 1396, 1289, 1188, 1154, 1083, 1018, 940, 835, and 796;
f) a differential scanning calorimetry curve exhibiting peaks at about 92.7° C., 120.1° C., and 125.9° C.;
g) a differential scanning calorimetry curve exhibiting peaks at about 68.0° C., 95.3° C., 115.8° C., and 126.3° C.; and
h) an endothermic maximum at about 310° C. measured using differential scanning calorimetry.

2. The crystalline polymorph of claim 1, wherein the polymorph is designated as Form Ia, and wherein the polymorph has an X-ray powder diffraction pattern exhibiting one or more of the approximate interplanar spacing peaks selected from the group consisting of 4.946, 7.118, 7.785, 8.238, 9.229, 9.822, 13.4000, 15.271, 15.658, 16.128, 16.459, 17.286, 18.088, 17.452, 18.889, 19.490, 19.837, 21.456, 22.658, 23.168, 23.811, 24.691, 28.436, and 29.609.

3. The crystalline polymorph of claim 2, wherein the polymorph form Ia is present in a purity of at least about 90%.

4. The crystalline polymorph of claim 1, wherein the polymorph is designated as Form Ib, and wherein the polymorph has an X-ray powder diffraction pattern exhibiting one or more of the approximate interplanar spacing peaks selected from the group consisting of 4.811, 8.316, 9.542, 10.047, 13.189, 14.946, 15.973, 17.219, 18.162, 21.814, 22.260, 23.087, 23.351, 24.518, 25.456, 26.846, 28.376, 29.648, 30.509, 31.226, and 32.328.

5. The crystalline polymorph of claim 4, wherein the polymorph form Ib is present in a purity of at least about 90%.

6. The crystalline polymorph of claim 1, wherein the polymorph is designated as Form II, and wherein the polymorph has an X-ray powder diffraction pattern exhibiting one or more of the approximate interplanar spacing peaks selected from the group consisting of 4.794, 7.020, 7.747, 8.104, 9.457, 11.483, 13.223, 15.010, 15.693, 16.943, 18.222, 19.552, 22.498, 23.003, and 29.490.

7. The crystalline polymorph of claim 6, wherein the polymorph form II is present in a purity of at least about 90%.

8. The crystalline polymorph of claim 1, wherein the polymorph is designated as Form Ib, and wherein the polymorph has an FTIR pattern exhibiting one or more of the approximate peaks selected from the group consisting of 3334, 3194, 1597, 1556, 1492, 1446, 1400, 1367, 1338, 1314, 1294, 1255, 1190, 1075, 1020, 992, 928, 835, 797, 748, and 733.

9. The crystalline polymorph of claim 1, wherein the polymorph is designated as Form II, and wherein the polymorph has an FTIR pattern exhibiting one or more of the approximate peaks selected from the group consisting of 332, 3207, 1555, 1499, 1443, 1396, 1289, 1188, 1154, 1083, 1018, 940, 835, and 796.

10. The crystalline polymorph of claim 1, wherein the polymorph has an endothermic maximum at about 310° C. measured using differential scanning calorimetry.

11. The crystalline polymorph of claim 1, wherein the polymorph is in the form of a hydrate.

12. The crystalline polymorph of claim 11, wherein the hydrate comprises about 10% to about 40% by weight water.

13. The crystalline polymorph of claim 11, wherein the hydrate is stable for a time of at least about 1 month when stored at a temperature of about 25° C. and a relative humidity of about 60% such that there is no significant additional water uptake by the hydrate.

14. A method of preparing the crystalline polymorph of claim 1, comprising forming a solution of the compound of Formula (1) and a polar solvent, combining at least a portion of the formed solution with a non-polar solvent, and isolating a solid, crystalline material that is a polymorph of the compound of Formula (1).

15. The method of claim 14, wherein the polar solvent comprises water and an alcohol.

16. The method of claim 14, wherein the non-polar solvent is selected from the group consisting of methylethylketone, methyl isobutylketone, tetrahydrofuran, and combinations thereof.

17. The method of claim 14, further comprising, prior to said combining step, removing any impurities from the compound of Formula (1) by combining the compound with activated carbon.

18. The method of claim 17, wherein the crystalline polymorph is a specific form, and wherein the isolated, specific form of the polymorph has a purity of at least about 90%.

19. A pharmaceutical composition comprising: a pharmaceutically acceptable carrier; and a therapeutically effective amount of a polymorph of the compound of Formula (1), or a pharmaceutically acceptable prodrug or pharmaceutically active metabolite thereof. 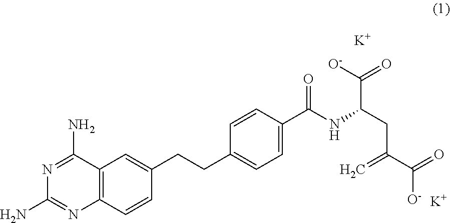
wherein the polymorph is a crystalline compound characterized by one or more of the following:
a) one or more of the approximate X-ray powder diffraction interplanar spacing peaks selected from the group consisting of 4.946, 7.118, 7.785, 8.238, 9.229, 9.822, 13.4000, 15.271, 15.658, 16.128, 16.459, 17.286, 18.088, 17.452, 18.889, 19.490, 19.837, 21.456, 22.658, 23.168, 23.811, 24.691, 28.436, and 29.609;
b) one or more of the approximate X-ray powder diffraction interplanar spacing peaks selected from the group consisting of 4.811, 8.316, 9.542, 10.047, 13.189, 14.946, 15.973, 17.219, 18.162, 21.814, 22.260, 23.087, 23.351, 24.518, 25.456, 26.846, 28.376, 29.648, 30.509, 31.226, and 32.328;
c) one or more of the approximate X-ray powder diffraction interplanar spacing peaks selected from the group consisting of 4.794, 7.020, 7.747, 8.104, 9.457, 11.483, 13.223, 15.010, 15.693, 16.943, 18.222, 19.552, 22.498, 23.003, and 29.490;
d) one or more of the approximate FTIR peaks selected from the group consisting of 3334, 3194, 1597, 1556, 1492, 1446, 1400, 1367, 1338, 1314, 1294, 1255, 1190, 1075, 1020, 992, 928, 835, 797, 748, and 733;
e) one or more of the approximate FTIR peaks selected from the group consisting of 332, 3207, 1555, 1499, 1443, 1396, 1289, 1188, 1154, 1083, 1018, 940, 835, and 796;
f) a differential scanning calorimetry curve exhibiting peaks at about 92.7° C., 120.1° C., and 125.9 ° C.;
g) a differential scanning calorimetry curve exhibiting peaks at about 68.0° C., 95.3° C., 115.8° C., and 126.3° C.; and
h) an endothermic maximum at about 310° C. measured using differential scanning calorimetry.

20. A method for treating a condition selected from the group consisting of abnormal cell proliferation, inflammation, asthma, and arthritis, said method comprising administering to a subject in need of treatment a therapeutically effective amount of a polymorph of the compound of Formula (1), or a pharmaceutically acceptable prodrug or pharmaceutically active metabolite thereof 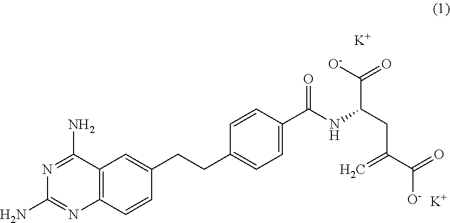
wherein the polymorph is a crystalline compound characterized by one or more of the following:
a) one or more of the approximate X-ray powder diffraction interplanar spacing peaks selected from the group consisting of 4.946, 7.118, 7.785, 8.238, 9.229, 9.822, 13.4000, 15.271, 15.658, 16.128, 16.459, 17.286, 18.088, 17.452, 18.889, 19.490, 19.837, 21.456, 22.658, 23.168, 23.811, 24.691, 28.436, and 29.609;
b) one or more of the approximate X-ray powder diffraction interplanar spacing peaks selected from the group consisting of 4.811, 8.316, 9.542, 10.047, 13.189, 14.946, 15.973, 17.219, 18.162, 21.814, 22.260, 23.087, 23.351, 24.518, 25.456, 26.846, 28.376, 29.648, 30.509, 31.226, and 32.328;
c) one or more of the approximate X-ray powder diffraction interplanar spacing peaks selected from the group consisting of 4.794, 7.020, 7.747, 8.104, 9.457, 11.483, 13.223, 15.010, 15.693, 16.943, 18.222, 19.552, 22.498, 23.003, and 29.490;
d) one or more of the approximate FTIR peaks selected from the group consisting of 3334, 3194, 1597, 1556, 1492, 1446, 1400, 1367, 1338, 1314, 1294, 1255, 1190, 1075, 1020, 992, 928, 835, 797, 748, and 733;
e) one or more of the approximate FTIR peaks selected from the group consisting of 332, 3207, 1555, 1499, 1443, 1396, 1289, 1188, 1154, 1083, 1018, 940, 835, and 796;
f) a differential scanning calorimetry curve exhibiting peaks at about 92.7° C., 120.1° C., and 125.9° C.;
g) a differential scanning calorimetry curve exhibiting peaks at about 68.0° C., 95.3° C., 115.8° C., and 126.3° C.; and
h) an endothermic maximum at about 310° C. measured using differential scanning calorimetry.

21. The method of claim 20, comprising administering the polymorph in combination with at least one further active agent.

22. The method of claim 21, wherein the at least one further active agent comprises methotrexate.

23. The method of claim 22, wherein the condition is arthritis.

24. The method of claim 23, wherein the condition is rheumatoid arthritis.
